(19) United States
(12) Patent Application Publication
(10) Pub. No.: US 2022/0331586 A1
(43) Pub. Date: Oct. 20, 2022

(54) NEUROSTIMULATION RESPONSE AND CONTROL

(71) Applicant: Medtronic, Inc., Minneapolis, MN (US)

(72) Inventors: Sarah J. Offutt, Golden Valley, MN (US); Julia P. Slopsema, Minneapolis, MN (US); Katelynn M. Johnson, New Brighton, MN (US); Lisa M. Jungbauer Nikolas, Lino Lakes, MN (US); Lance Zirpel, Lino Lakes, MN (US); Leonid M. Litvak, Los Angeles, CA (US); Simeng Zhang, Golden Valley, MN (US); Katie C. Bittner, White Bear Lake, MN (US)

(21) Appl. No.: 17/658,984

(22) Filed: Apr. 12, 2022

Related U.S. Application Data
(60) Provisional application No. 63/175,499, filed on Apr. 15, 2021.

(51) Int. Cl. A61N 1/36; A61N 1/05

(52) U.S. Cl. CPC A61N 1/36007; A61N 1/0551

(57) ABSTRACT

An example method includes delivering, via an electrical stimulation device, one or more electrical stimulation signals to a patient, sensing one or more stimulation-evoked signals that are evoked by stimulation of nerves or muscles of the patient due to the delivery of the one or more electrical stimulation signals, determining a quality of the one or more stimulation-evoked signals, and outputting, based on the quality of the one or more sensed stimulation-evoked signals being below a quality threshold, one or more instructions to improve the quality of one or more subsequent stimulation-evoked signals.

This application claims the benefit of U.S. Provisional Application Ser. No. 63/175,499, filed Apr. 15, 2021, the entire content of which is incorporated herein by reference.

### TECHNICAL FIELD

This disclosure generally relates to medical devices, and more specifically, electrical stimulation.

### BACKGROUND

Electrical stimulation devices, sometimes referred to as neurostimulators or neurostimulation devices, may be external to or implanted within a patient, and configured to deliver electrical stimulation therapy to various tissue sites to treat a variety of symptoms or conditions such as chronic pain, tremor, multiple sclerosis, stroke, spinal cord injury, neuropathy, Parkinson's disease, epilepsy, or other neurological disorders, bladder dysfunction such as retention, overactive bladder, urgency, urgency frequency, urinary incontinence, bladder incontinence, fecal incontinence, intractable constipation, pelvic pain, irritable bowel syndrome, inflammatory bowel disease, interstitial cystitis, neurogenic bowel/bladder, sexual dysfunction, obesity, or gastroparesis. An electrical stimulation device may deliver electrical stimulation therapy via electrodes, e.g., carried by one or more leads, positioned proximate to target locations associated with the brain, the spinal cord, nerves of the pelvis and pelvic floor, tibial nerves, peripheral nerves, the gastrointestinal tract, or elsewhere within a patient. Stimulation proximate the spinal cord, proximate the sacral nerves, within the brain, and proximate peripheral nerves is often referred to as spinal cord stimulation (SCS), sacral neuromodulation (SNM), deep brain stimulation (DBS), and peripheral nerve stimulation (PNS), respectively.

A physician or clinician, or patient, may select values for a number of programmable stimulation parameters in order to define the electrical stimulation therapy to be delivered by the implantable stimulator to a patient. For example, the physician or clinician may select one or more electrodes, polarities of selected electrodes, a voltage or current amplitude, a pulse width, a pulse frequency, a cycling, and a duration of stimulation as stimulation parameters. A set of therapy stimulation parameters, such as a set including electrode combination or configuration, electrode polarity, amplitude, pulse width, pulse shape, pulse frequency or pulse rate, or cycling may be referred to as a therapy program in the sense that they define the electrical stimulation therapy to be delivered to the patient.

### SUMMARY

In one example, this disclosure describes a method that includes delivering, via an electrical stimulation device, one or more electrical stimulation signals to a patient for modulating bladder and/or bowel control; sensing one or more stimulation-evoked signals that are evoked by stimulation of nerves or muscles of the patient due to the delivery of the one or more electrical stimulation signals; determining a quality of the one or more stimulation-evoked signals; and outputting, based on the quality of the one or more sensed stimulation-evoked signals being below a quality threshold, one or more instructions to improve the quality of one or more subsequent stimulation-evoked signals.

In another example, this disclosure describes a system includes at least one electrode configured to deliver the electrical stimulation to a patient; and a device including processing circuitry configured to: deliver one or more electrical stimulation signals to a patient for modulating bladder and/or bowel control; sense one or more stimulation-evoked signals that are evoked by stimulation of nerves or muscles of the patient due to the delivery of the one or more electrical stimulation signals; determine a quality of the one or more stimulation-evoked signals; and output, based on the quality of the one or more sensed stimulation-evoked signals being below a quality threshold, one or more instructions to improve the quality of one or more subsequent stimulation-evoked signals.

In another example, this disclosure describes a computer readable medium including instructions that when executed cause one or more processors to: cause an electrical stimulation device to deliver one or more electrical stimulation doses to a patient for modulating bladder and/or bowel control; receive one or more sensed stimulation-evoked signals that are evoked by stimulation of nerves or muscles of the patient due to the delivery of the one or more electrical stimulation signals; determine a quality of the one or more sensed stimulation-evoked signals; and output, based on the quality of the one or more sensed stimulation-evoked signals being below a quality threshold, one or more instructions to improve the quality of one or more subsequent stimulation-evoked signals.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating an example system that includes an implantable medical device (IMD) in the form of a neurostimulation device configured to deliver sacral neuromodulation (SNM), an external programmer, and one or more sensing devices in accordance with one or more techniques of this disclosure.

FIG. 2A is a block diagram illustrating an example of an IMB in the form of a neurostimulation device, in accordance with one or more techniques of this disclosure.

FIG. 2B is a block diagram illustrating an example of an IMB in the form of a neurostimulation device, in accordance with one or more techniques of this disclosure.

FIG. 3 is a block diagram illustrating an example of an external programmer suitable for use with the IMD of FIG. 2, in accordance with one or more techniques of this disclosure.

FIG. 4 is a flow diagram illustrating an example method of operation, in accordance with one or more techniques of this disclosure.

FIG. 5 is a plot of an example stimulation-evoked signal, in accordance with one or more techniques of this disclosure.

FIG. 6 is a plot of another example stimulation-evoked signal, in accordance with one or more techniques of this disclosure.

FIG. 7 is a plot of another example stimulation-evoked signal, in accordance with one or more techniques of this disclosure.

FIG. 8 is a plot of another example stimulation-evoked signal, in accordance with one or more techniques of this disclosure.

FIG. 9 is a plot of another example stimulation-evoked signal, in accordance with one or more techniques of this disclosure.

FIG. 10 is a plot of another example stimulation-evoked signal, in accordance with one or more techniques of this disclosure.

FIG. 11 is a plot of another example accompanying signal, in accordance with one or more techniques of this disclosure.

FIG. 12 is a plot of another example accompanying signal, in accordance with one or more techniques of this disclosure.

FIG. 13 is a plot of example accompanying signals, in accordance with one or more techniques of this disclosure.

FIG. 14 is a plot of example accompanying signals, in accordance with one or more techniques of this disclosure.

FIG. 15 is a plot of an example stimulation-evoked signal, in accordance with one or more techniques of this disclosure.

FIG. 16 is a plot of another example stimulation-evoked signal, in accordance with one or more techniques of this disclosure.

FIG. 17 is a plot of another example stimulation-evoked signal, in accordance with one or more techniques of this disclosure.

FIG. 18 is a plot of another example stimulation-evoked signal, in accordance with one or more techniques of this disclosure.

FIG. 19 is a plot of another example composite stimulation-evoked signal, in accordance with one or more techniques of this disclosure.

### DETAILED DESCRIPTION

Electrical stimulation therapy, e.g., sacral nerve stimulation, tibial nerve stimulation, and/or other types of invasive or noninvasive neuromodulation, may provide bladder and/or bowel dysfunction therapy, pain relief and/or other therapeutic benefits. Electrical stimulation may evoke a response (e.g., a signal) such as a neural response of one or more nerves and contractions, activity, and/or electrical activity of one or more muscles. For example, stimulation of sacral nerves through electrical leads implanted near sacral nerves via sacral neuromodulation may evoke a neural response in adjacent nerves, muscle contractions within the pelvic floor, and distal contractions in the foot. The neural response in nerves and activation/contraction of muscles evoked by electrical stimulation may be captured (e.g., or detected, sensed, measured, and the like) as a stimulation-evoked signal that may be a composite signal comprising two or more distinct signals generated from one or more signal sources. For examples, a composite stimulation-evoked signal may comprise two or more stimulation-evoked signals generated by one or more signal sources in response to the electrical stimulation, e.g., two or more signals may come from one signal source or more than one signal source. A sensed composite stimulation-evoked signal may be a composite of signals from one or more nerves, one or more muscles, or at least one muscle and/or at least one nerve captured concurrently within a particular amount of time.

In some examples, a composite stimulation-evoked signal may be a composite of two or more stimulation-evoked signals from two or more sources, e.g., the two or more sources being two or more anatomical structures of the patient such as particular nerves and/or particular muscles. In other examples, a composite stimulation-evoked signal may be a composite of two or more stimulation-evoked signals from a single source, e.g., a single anatomical structure such as a particular nerve or particular muscle. For example, in response to stimulation, a nerve may generate both an evoked compound action potential (ECAP) signal and a reflex signal after a time delay. As another example, a muscle may activate and/or contract in response to stimulation, and over a period of time additional and/or a different subset of muscle fibers may contract and/or release, generating a first signal over a first period of time (corresponding to a first subset of muscle fibers activating/contracting) and a second signal over a second period of time (corresponding to a second subset of muscle fibers activating/contracting). In some examples, a composite stimulation-evoked signal may be a composite of two or more stimulation-evoked signals of the same type, or a different type, e.g., two ECAPs, an ECAP and an electromyogram (EMG), or the like.

In some examples, a stimulation-evoked signal (or stimulation-evoked composite signal) may be a “direct stimulation-evoked signal” that is directly evoked, e.g., evoked by a signal source (e.g., nerve or muscle) in response to stimulation of that same signal source. In other examples, a stimulation-evoked signal (or stimulation-evoked composite signal) may be an “indirect stimulation-evoked signal” that is indirectly evoked, e.g., evoked by a signal source (e.g., nerve or muscle) in response to stimulation of a different part of the patient's anatomy. For example, a signal evoked by a distal contraction of the patient's foot, where the distal contraction is in response to stimulation of a sacral nerve, is an indirect stimulation-evoked signal. Throughout the disclosure, the term “stimulation-evoked signal” encompasses any or all of direct, indirect, single, and/or composite stimulation-evoked signals unless further specified.

A stimulation-evoked signal may include one or more features that may indicate one or more aspects of electrical stimulation therapy delivery, such as electrical stimulation parameters, stimulation timing, a positioning of electrical lead(s) that provides effective therapy, e.g., electrical lead placement that improves symptoms and/or disease systems, and the like. Stimulation-evoked signals, or a lack thereof, e.g., a lack of activation/response/contraction in response to electrical stimulation, may indicate a placement of the electrical lead that does not provide effective therapy, e.g., poor electrical lead placement and subsequent therapy. Capturing and processing stimulation-evoked signals in an operating room, a clinic, at home, or in other environments presents several challenges.

For example, the patient may move and/or be active during electrical stimulation delivery and/or sensing of stimulation-evoked signals. For example, it may be more difficult to control and/or monitor patient behavior in a non-clinical environment, such as an electrical stimulation therapy session in an at home and/or remote (e.g., non-clinical) environment. Such movement may cause noise and/or artifacts in stimulation-evoked signals, may change the response of one or more signal sources that would otherwise respond differently, and may confound and/or reduce the reliability and/or trustworthiness of information derived from or based on the stimulation-evoked signals.

For example, patient movement may cause the leads delivering electrical stimulation therapy to move relative to the nerve and/or muscle being stimulated and/or move relative to a remote signal source (e.g., a different nerve and/or muscle). Changing distance between one or more electrodes delivering the stimulation and the stimulation target (e.g., nerve and/or muscle) may change the response of the stimulation target and/or one or more other signal sources, and changing distance between a sensor (which may be the same one or more electrode) and the signal sources may change the received stimulation-evoked signal (e.g., signal strength). Such changes may cause noise and/or artifacts in sensed stimulation-evoked signals, thereby increasing the difficulty, and reducing the reliability, of determining the responses of one or more signal sources to the delivered electrical stimulation signals, and/or the therapy efficacy, based on the stimulation-evoked signals.

Additionally, patient posture during electrical stimulation delivery and/or sensing of stimulation-evoked signals may change and/or alter the response of the electrical stimulation target and/or other signal sources. Patient posture may include a position of the patient, e.g., sitting, standing, lying down, and the like. Patient posture may also include an amount and/or type of patient movement, e.g., resting, running, walking, changing position, and the like. Additionally, patient posture may also include a patient state, e.g., a physiological state such as a full and/or empty bladder, an increased heart rate, an increased body temperature, and the like. For example, a nerve and/or muscle may have a greater or lesser response (e.g., the amplitude of an electrical signal delivered to nerve or muscle may be greater or lesser) depending on the patient's particular position and/or patient movement because of the position/movement itself rather than a changing electrode distance. For example, a muscle that is contracted due to patient position, and/or in a state of contracting/relaxing due to patient movement, may have a different response to electrical stimulation than is otherwise expected and/or desired.

Additionally, a nerve and/or muscle may have a response that is not evoked by the stimulation, e.g., a non-stimulation-evoked and/or spurious response. A spurious response of a signal source may cause a spurious signal which may be sensed along with any stimulation-evoked signals, which may in turn cause an incorrect determination regarding signal source responses (e.g., to stimulation) and/or the efficacy of the stimulation therapy, or may cause uncertainty (e.g., reduced reliability and/or trustworthiness) regarding signal source responses and/or the efficacy of the stimulation therapy. For example, a spurious signal may cause a “false positive,” e.g., a conclusion that the signal source responded to the electrical stimulation, a “false negative,” e.g., by masking stimulation-evoked signals from other signal sources that may be overwhelmed by the spurious signal preventing determination that those signal sources had a response.

In accordance with one or more techniques of this disclosure, example electrical stimulation systems and example techniques may determine a quality of stimulation-evoked signals, e.g., the reliability and/or trustworthiness of stimulation-evoked signals and/or an amount of uncertainty in determinations and information derived from and/or based on the stimulation-evoked signals. The systems and techniques disclosed herein may determine the quality of the stimulation-evoked signals (e.g., any or all of direct, indirect, single, and/or composite stimulation-evoked signals) and whether the stimulation-evoked signals may be used, e.g., whether the determinations based on the stimulation-evoked signals are reliable and/or trustworthy.

In some examples, the system and techniques disclosed herein may determine the quality of stimulation-evoked signals based on determining features of the stimulation-evoked signals and/or based on additional information such as one or more accompanying signals from one or more other sensors that are sensed at or near the time of delivery of electrical stimulation signals and/or sensing of the stimulation-evoked signals. For example, the quality of a stimulation-evoked signal may be determined based on a stimulation-evoked signal alone, based on an accompanying signal alone, or based on both. Examples of accompanying signals may include a signal from an accelerometer configured to indicate patient movement and/or posture, a signal from a heart rate monitor configured to indicate the heart rate of the patient (e.g., from which patient movement may be determined), a signal from a pressure sensor configured to indicate the fullness of the patient's bladder and/or voiding events (e.g., from which a responsivity of certain signal sources, such as a detrusor muscle, may be inferred), or any other signal from any other sensor configured to determine a patient posture at or near the time of the delivery of electrical stimulation signals and/or sensing of the stimulation-evoked signals. Generally, accompanying signals may be sensor-generated signals indicative of body movement or non-lead or non-electrode sensed signals, e.g., sensed by something other than an electrode of a lead (such as leads and electrodes described below).

For example, a system may determine a quality of a stimulation-evoked signal based on a signal-to-noise ratio (SNR) of the stimulation-evoked signal, a determination that the stimulation-evoked signal includes one or more signal artifacts, the strength (e.g., amplitude and/or length of time) of the stimulation-evoked signal, the spectral content of the stimulation-evoked signal, any suitable signal feature determined, for example, by signal processing, or any other suitable signal feature. Alternatively or additionally, a system may determine the quality of a stimulation-evoked signal based on an accompanying signal indicating a particular patient posture such as information and/or data from an accelerometer, e.g., an accelerometer of the patient's phone, the patient's programming device and/or implanted device, a patient's wearable device, and the like. In an example relating to sacral nerve stimulation, a system may determine the quality of a stimulation-evoked signal based on an accompanying signal from a pressure sensor indicating a fullness of the patient's bladder and/or whether any voiding events occurred during the course of delivery of an electrical stimulation therapy program and sensing of the stimulation-evoked signal. For example, an accompanying signal indicating bladder pressure information may indicate a responsivity of nerves and/or muscles to electrical stimulation, e.g., a full or empty bladder may change whether nerves and/or muscles related to the bladder (e.g., sacral nerve, detrusor muscle, etc.), respond to electrical stimulation and/or an amount of the response. In some examples, whether certain signal sources respond, and the amount of the response, may be known and/or expected based on patient posture, and accompanying signals may indicate the particular posture from which the expected responses of signal sources may be inferred.

In some examples, the systems and techniques disclosed herein may output one or more instructions to improve the quality of the one or more stimulation-evoked signals (e.g., instructions for causing improvement in the quality of one or more subsequent stimulation-evoked signals). For example, a system may output instructions directing the patient to be in a particular posture, e.g., to stop moving, to sit, stand, or lie down, and the like, and/or directing electrical stimulation to occur after a patient action and/or event, such as voiding event. The system may output instructions to direct the patient, clinician, and/or user of the system to change one or more electrical stimulation therapy parameters, e.g., a particular combination of electrodes, polarities of selected electrodes, a voltage or current amplitude, a pulse width, a pulse frequency, and the like. In some examples, a system may output instructions to automatically change one or more electrical stimulation therapy parameters, e.g., to cause a patient programmer device to change one or more of the electrical stimulation therapy parameters.

In some examples, a stimulation-evoked signal may comprise one or more EMG. In some examples, the stimulation-evoked signal may comprise more than an EMG, e.g., a compound action potential such as an ECAP, a surface EMG, an MMG, a network excitability, and/or multiple signals of differing signal type evoked by one or more signal sources. In some examples, signal sources may include nerves such as sacral nerves, dorsal and ventral rami of sacral nerves, pudendal nerves, sciatic nerves, saphenous nerves, nerves in the sacral plexus, pelvic nerves, pelvic plexus nerves, pelvic splanchnic nerves, inferior hypogastric plexus nerves, lumbosacral trunk nerves, where the lumbosacral trunk joins sacral nerves, any sympathetic nerve fibers in the sympathetic chain of any of the above nerves or other nerves. In some examples, signal sources may include muscles such as an external anal sphincter muscle, coccygeus muscle, levator ani muscle group, bulbocavernosus and/or bulbospongiosus muscle, gluteal muscles, e.g., gluteal maximus, gluteal medius, and gluteal minimus, perineal muscles, ischiocavernosus muscles, puborectalis muscles, piriformis muscles, or any other muscles.

In some examples, a stimulation-evoked signal may be a composite stimulation-evoked signal, e.g., two or more stimulation-evoked signals generated by one or more signal sources in response to the one or more electrical stimulation signals may be sensed as a composite stimulation-evoked signal. For example, a medical device may output one or more electrical stimulation signals (e.g., waveforms) via stimulation electrodes on a lead, and sensing electrodes on the same lead (e.g., which may be the same electrodes as the stimulation electrodes, or different electrodes from the stimulation electrodes) or on a different lead may sense one or more neural responses and/or one or more muscle activation/contraction responses as one or more stimulation-evoked signals. In some examples, one or more sensing electrodes may sense a composite stimulation-evoked signal that is a composite of signals generated by one or more signal sources, e.g., muscles and/or nerves, in response to the delivered electrical stimulation signals. In some examples, the signals generated by two more signal sources may be stimulation-evoked signals. For example, the sensed composite stimulation-evoked signal may be a composite of signals from one or more muscles, one or more nerves, one or more nerve fibers of, or within, a nerve, or at least one muscle and at least one nerve (or nerve fiber) captured concurrently within a particular amount of time. The particular amount of time may be an amount of time starting when the electrical stimulation begins or ends, and ending after a predetermined amount of time has passed, or ending based on the composite stimulation-evoked signal, one or more of the constituent signals of the composite stimulation-evoked signal, or some other trigger such as a physiological response or patient-input response is received, or ending based on other criteria. In some examples, a composite stimulation-evoked signal may be a composite of two or more signals generated by single signal source, e.g., at different times and captured within a particular amount of time. For example, delivery of an electrical stimulation signal may cause multiple responses from a single signal source, e.g., a muscle or nerve, and each response of the signal source may generate a signal (e.g., a stimulation-evoked signal).

In some examples, a composite stimulation-evoked signal may include signal features indicative of the responses of one or more signal sources (e.g., nerves or muscles) that occur over a relatively long period of time, e.g., more than 5 milliseconds (ms), more than 10 ms, more than 20 ms, etc. In other words, a composite stimulation-evoked signal may contain information relating to the efficacy of electrical stimulation therapy from the responses of the signal source(s) and may occur over a relatively long period of time (e.g., a relatively long signal capture time window). For example, different signal sources may have different response times, e.g., neural responses versus muscle contractions, and the different sources may be located at different distances from both the electrical stimulation source (e.g., an electrode of a lead) and a sensor (e.g., which may be the same and/or a different electrode on the same and/or different lead, or a different sensor located within and/or external to the patient's body). In order to capture at least a portion or substantially all of each of the stimulation-evoked signals from the different signal sources, the signal capture time window may be longer than any single stimulation-evoked signal because of the varying response times, temporal signal lengths, and signal source distances. In some examples, the timing of the sensing/receipt of stimulation-evoked signals may depend on how fast a particular signal source activates, e.g., adjacent nerves may be the fastest (e.g., shortest response time) and a muscle or any a post-synaptic neural activation may be slower (e.g., have a longer response time). For example, a direct stimulation-evoked nerve response (e.g., neural signal) may be generated within about 3 ms after stimulation, a muscle signal (e.g., generated by a muscle response such as a contraction, muscle activity, muscle electrical activity, or the like) may be generated within about 15 ms after stimulation, and a reflex muscle response (e.g., contraction, activity, electrical activity, or the like) may be generated within about 75 ms after stimulation. The timing of the sensing/receipt of stimulation-evoked signals may also depend on how close the signal source is to the sensing/capturing electrode, e.g., it takes some time for the signal to get to the electrode. In some examples, the composite stimulation-evoked signal may include a plurality of stimulation-evoked signals that “arrive” at a lead over a period of time and are received at least partially overlapping in time. In some examples, such component stimulation-evoked signals of the sensed/received composite stimulation-evoked signal may be distinguishable from each other via their respective signal features.

In some examples, a composite stimulation-evoked signal may provide more complete information regarding stimulation therapy efficacy, e.g., as opposed to capturing an individual signal and/or stimulation-evoked signal from one or more signal sources. For example, the ensemble of signal sources may respond differently than the sum of individual signal sources, e.g., there may be interactions between the source and/or sources generating the plurality of stimulation-evoked signals, and systems and/or techniques disclosed may include a signal capture time window that is long enough to capture the ensemble response as a composite stimulation-evoked signal.

In some examples, a composite stimulation-evoked signal may comprise one or more compound action potentials, e.g., an evoked compound action potential (ECAP). In some examples, the composite stimulation-evoked signal may comprise more than a compound action potential, e.g., one or more of an ECAP, an EMG or surface EMG, mechanomyography (e.g., an MMG), a network excitability, and/or multiple signals of differing signal type evoked by one or more signal sources. In some examples, the composite stimulation-evoked signal may be a combination of any and/or all of the various signal sources. For example, an electrical stimulation signal may cause a nerve and/or muscle proximate to the stimulation signal to generate a response and other nerves or muscles, not necessarily proximate to the stimulation signal, may also generate responses. In some examples, an electrical stimulation signal may cause a proximate nerve to respond and/or activate the muscle innervated by the proximate nerve and causing the muscle to respond and/or directly activate one or muscles and causing those one or more muscles to response. In some examples, the electrical stimulation signal may be applied to the spinal cord, or may be applied proximate to the spinal cord, which may respond with a reflex and/or reflex signal, e.g., one or more nerve fibers may evoke one or more reflexes and/or reflex signals, which may be stimulation-evoked signals. In some examples, a reflex and/or reflex signal may be elicited and/or caused from a nerve proximate to the spinal cord, e.g., via stimulation applied to such proximate nerve or stimulation applied to, or proximate to, the spinal cord which then causes such proximate nerve to response to elicit the reflex and/or reflex signal. The composite stimulation-evoked signal may be a composite of signals from any of the multiple signal sources.

Systems and methods for sensing and determining a quality, reliability, and/or trustworthiness of stimulation-evoked signals are described herein. The system may include a stimulator system that interacts with a stimulator programmer. Various examples are discussed relative to one or more stimulation devices. It is recognized that the stimulation devices may include features and functionality in addition to electrical stimulation. Many of these additional features are expressly discussed herein. A few example features include, but are not limited to, different types of sensing capabilities and different types of wireless communication capabilities. For ease of discussion, the present disclosure does not expressly recite every conceivable combination of the additional features, such as by repeating every feature each time different examples and uses of the stimulation devices are discussed.

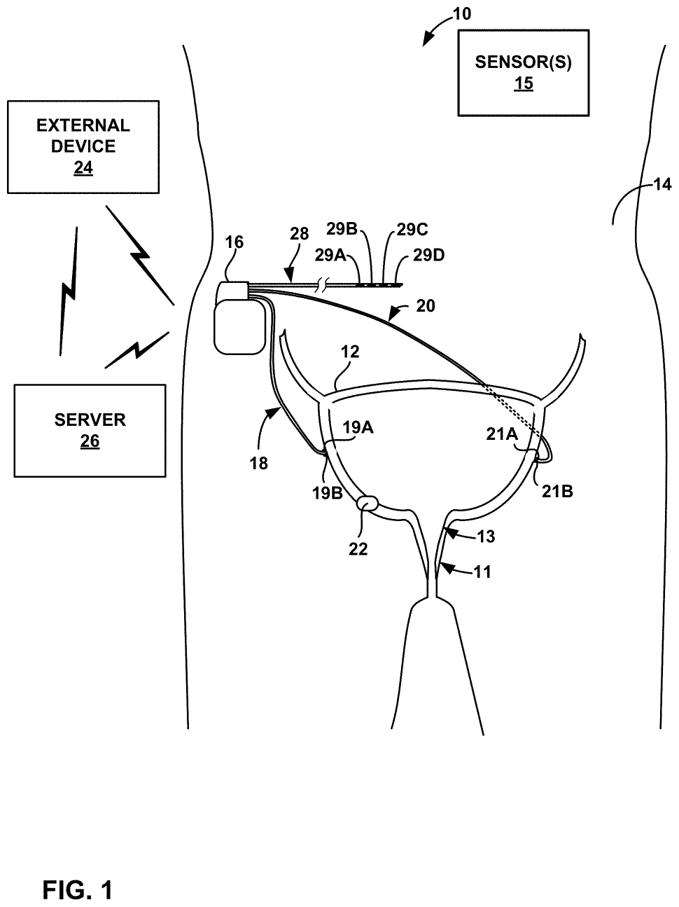

FIG. 1 is a conceptual diagram illustrating an example system 10 that includes an implantable medical device (IMD 16) in the form of a neurostimulation device configured to deliver sacral neuromodulation (SNM), an external programmer, and one or more sensing devices in accordance with one or more techniques of this disclosure. Although described with reference to IMD 16, system 10 may additionally or alternatively include an external medical device configured to perform the functions of IMD 16, e.g., an external neurostimulator such as used for trialing and configured to deliver electrical stimulation through any or all of leads 18, 20, 28, or percutaneous leads (not shown). In some examples, system 10 may determine one or more stimulation setting(s) and manage delivery of neurostimulation to patient 14, e.g., to manage bladder and/or bowel dysfunction, such as retention, overactive bladder, urgency, urgency frequency, urinary incontinence, bladder incontinence, bowel incontinence, fecal incontinence. As shown in the example of FIG. 1, therapy system 10 includes an implantable medical device (IMD) 16 (e.g., an example medical device), which is coupled to leads 18, 20, and 28 and sensor 22. System 10 also includes an external device 24, which is configured to communicate with IMD 16 via wireless communication. System 10 also includes server 26 which may be one or more servers in a cloud computing environment. Server 26 may be configured to communicate with external device 24 and/or IMD 16 via wireless communication through a network access point (not shown in FIG. 1) and may be collocated with external device 24 or may be located elsewhere, such as in a cloud computing data center. IMD 16 generally operates as a therapy device that delivers neurostimulation (e.g., electrical stimulation in the example of FIG. 1) to, for example, a target tissue site proximate a spinal nerve, a sacral nerve, a pudendal nerve, a dorsal nerve of the penis, a dorsal nerve of the clitoris, a tibial nerve, a saphenous nerve, an inferior rectal nerve, a perineal nerve, or other pelvic nerves, branches of any of the aforementioned nerves, roots of any of the aforementioned nerves, ganglia of any of the aforementioned nerves, or plexuses of any of the aforementioned nerves. IMD 16 provides electrical stimulation to patient 14 by generating and delivering a programmable electrical stimulation signal (e.g., in the form of electrical pulses or an electrical signal) to a target a therapy site near lead 28 and, more particularly, near electrodes 29A-29D (collectively referred to as “electrodes 29”) disposed proximate to a distal end of lead 28.

IMD 16 may be surgically implanted in patient 14 at any suitable location within patient 14, such as near the pelvis. In some examples, IMD 16 may be implanted in a subcutaneous location in the side of the lower abdomen or the side of the lower back or upper buttocks. IMD 16 has a biocompatible housing, which may be formed from titanium, stainless steel, a liquid crystal polymer, or the like. The proximal ends of leads 18, 20, and 28 are both electrically and mechanically coupled to IMD 16 either directly or indirectly, e.g., via respective lead extensions. Electrical conductors disposed within the lead bodies of leads 18, 20, and 28 electrically connect sense electrodes (e.g., electrodes 19A, 19B, 21A, 21B, 29A, 29B, 29C, and 29D) and stimulation electrodes, such as electrodes 29, to sensing circuitry and a stimulation delivery circuitry (e.g., a stimulation generator) within IMD 16. In the example of FIG. 1, leads 18 and 20 carry electrodes 19A, 19B (collective referred to as “electrodes 19”) and electrodes 21A, 21B (collectively referred to as “electrodes 21”), respectively. As described in further detail below, electrodes 19 and 21 may be positioned for sensing an impedance of bladder 12, which may increase as the volume of urine within bladder 12 increases. In some examples, system 10 may include electrodes (such as electrodes 19 and 21), a strain gauge, one or more accelerometers, ultrasound sensors, optical sensors, or any other sensor, any of which be a sensor 15 as shown in FIG. 1 and further described below. In some examples, sensors 15 may be configured to gather information relating to the patient, such as detect contractions of bladder 12, pressure or volume of bladder 12, or any other indication of the fill cycle of bladder 12 and/or possible bladder dysfunctional states. In some examples, system 10 may use sensors 15 other than electrodes 19 and 21 for sensing information relating to the patient, such as bladder volume, cardiac response, chemical responses, or the like. A stimulation-evoked signal (e.g., any or all of direct, indirect, single, and/or composite stimulation-evoked signals) may include a cardiac signal and/or a chemical signal, and sensors 15 may include a cardiac sensor and/or a chemical sensor. System 10 may use the sensor 15 data for determining a quality of stimulation-evoked signals and/or determining stimulation program settings for a given patient. IMD 16 may communicate sensed data to server 26. In some examples, IMD 16 may communicate the sensor 15 data through external device 24. In other examples, IMD 16 may communicate the sensor 15 data to server 26 without communicating the sensor 15 data through external device 24.

In some examples, external device 24 may collect user input identifying a voiding event, perceived level of fullness, or any other indication of an event associated with the patient. The user input may be in the form of a voiding journal analyzed by external device 24, IMD 16 or server 26, or individual user inputs associated with respective voiding events, leakage, or any other event related to the patient. External device 24 may provide this user input to server 26.

One or more medical leads, e.g., leads 18, 20, and 28, may be connected to IMD 16 and surgically or percutaneously tunneled to place one or more electrodes carried by a distal end of the respective lead at a desired nerve or muscle site, e.g., one of the previously listed target therapy sites such as a tissue site proximate a spinal (e.g., sacral) or pudendal nerve. For example, lead 28 may be positioned such that electrodes 29 deliver electrical stimulation to a spinal, sacral or pudendal nerve to reduce a frequency and/or magnitude of contractions of bladder 12. Additional electrodes of lead 28 and/or electrodes of another lead may provide additional stimulation therapy to other nerves or tissues as well. In FIG. 1, leads 18 and 20 are placed proximate to an exterior surface of the wall of bladder 12 at first and second locations, respectively. In other examples of therapy system 10, IMD 16 may be coupled to more than one lead that includes electrodes for delivery of electrical stimulation to different stimulation sites within patient 14, e.g., to target different nerves.

In the example shown in FIG. 1, leads 18, 20, 28 are cylindrical. Electrodes 19, 21, 29 of leads 18, 20, 28, respectively, may be ring electrodes, segmented electrodes, partial ring electrodes or any suitable electrode configuration. Segmented and partial ring electrodes each extend along an arc less than 360 degrees (e.g., 90-120 degrees) around the outer perimeter of the respective lead 18, 20, 28. In some examples, segmented electrodes 29 of lead 28 may be useful for targeting different fibers of the same or different nerves to generate different physiological effects (e.g., therapeutic effects). In examples, one or more of leads 18, 20, 28 may be, at least in part, paddle-shaped (e.g., a “paddle” lead), and may include an array of electrodes on a common surface, which may or may not be substantially flat. In some examples, electrodes 19, 21, 29, may comprise a single electrode and an external ground, or be configured for use with one electrode and an external ground, e.g., such as may be used for peripheral nerve evaluation (PNE).

In some examples, one or more of electrodes 19, 21, 29 may be cuff electrodes that are configured to extend at least partially around a nerve (e.g., extend axially around an outer surface of a nerve). Delivering electrical stimulation via one or more cuff electrodes and/or segmented electrodes may help achieve a more uniform electrical field or activation field distribution relative to the nerve, which may help minimize discomfort to patient 14 that results from the delivery of electrical stimulation. An electrical field may define the volume of tissue that is affected when the electrodes 19, 21, 29 are activated. An activation field represents the neurons and/or muscles that will be activated by the electrical field in the neural tissue proximate to the activated electrodes.

The illustrated numbers and configurations of leads 18, 20, and 28 and electrodes carried by leads 18, 20, and 28 are merely exemplary. Other configurations, e.g., numbers and positions of leads and electrodes are also contemplated. For example, in other implementations, IMD 16 may be coupled to leads 18, 20, and 28, each of which may have a single electrode or multiple electrodes as shown, or additional leads or lead segments having one or more electrodes positioned at different locations proximate the spinal cord or in the pelvic region of patient 14. The additional leads may be used for delivering different stimulation therapies or other electrical stimulations to respective stimulation sites within patient 14 or for monitoring at least one physiological marker of patient 14.

In accordance with some examples of the disclosure, IMD 16 delivers electrical stimulation to at least one of a spinal nerve (e.g., a sacral nerve), a pudendal nerve, dorsal genital nerve, a tibial nerve, a saphenous nerve, an inferior rectal nerve, or a perineal nerve to provide a therapeutic effect that reduces or eliminates a dysfunctional state such as overactive bladder and/or fecal incontinence. The desired therapeutic effect may be an inhibitory physiological response related to voiding of patient 14, such as a reduction in bladder contraction frequency by a desired level or degree (e.g., percentage), a reduction in bladder afferent firing, altering a pelvic floor muscle/nerve response and/or status such as of the external urethral sphincter (EUS), levator ani nerve, external anal sphincter, and the like.

A stimulation program may define various parameters of the stimulation signal and electrode configuration which result in a predetermined stimulation intensity being delivered to the targeted nerve or tissue. In some examples, the stimulation program defines parameters for at least one of a current or voltage amplitude of the stimulation signal, a frequency or pulse rate of the stimulation, the shape of the stimulation signal, a duty cycle of the stimulation, a pulse width of the stimulation, a duty cycle of the stimulation ON/OFF periods, and/or the combination of electrodes 29 and respective polarities of one or more subsets of electrodes 29 used to deliver the stimulation. Together, these stimulation parameter values may be used to define the stimulation intensity (also referred to herein as a stimulation intensity level). In some examples, if stimulation pulses are delivered in bursts, a burst duty cycle also may contribute to stimulation intensity. Also, independent of intensity, a particular pulse width and/or pulse rate may be selected from a range suitable for causing the desired therapeutic effect after stimulation is terminated and, optionally, during stimulation. In addition, as described herein, a period during which stimulation is delivered may include on and off periods (e.g., a duty cycle or bursts of pulses) where even the short inter-pulse durations of time when pulses are not delivered are still considered part of the delivery of stimulation. A period during which system 10 withholds stimulation delivery is a period in which no stimulation program is active for IMD 16 (e.g., IMD 16 is not tracking pulse durations or inter-pulse durations that occur as part of the electrical stimulation delivery scheme). In addition to the above stimulation parameters, the stimulation may be defined by other characteristics, such as a time for which stimulation is delivered, a time for which stimulation is terminated, and times during which stimulation is withheld.

In certain embodiments, stimulation will be provided below or at sensory threshold of the patient, but sometimes, in order to evoke or maintain a certain physiological response (i.e. composite signal), the stimulation may be provided above the sensory threshold.

System 10 may also include an external device 24, as shown in FIG. 1. External device 24 may be an example of a computing device. In some examples, external device 24 may be a clinician programmer or patient programmer, such as patient programmer 300 described below. In some examples, external device 24 may be a device for inputting information relating to a patient. In some examples, external device 24 may be a wearable communication device, with a therapy request input integrated into a key fob or a wristwatch, handheld computing device, tablet, smart phone, computer workstation, or networked computing device. External device 24 may include a user interface that is configured to receive input from a user (e.g., patient 14, a patient caretaker or a clinician). In some examples, the user interface includes, for example, a keypad and a display, which may for example, be a liquid crystal display (LCD) or light emitting diode (LED) display. In some examples, the user interface may include a turnable knob or a representation of a turnable knob. The keypad may take the form of an alphanumeric keypad or a reduced set of keys associated with particular functions. External device 24 may additionally or alternatively include a peripheral pointing device, such as a mouse, via which a user may interact with the user interface. In some examples, a display of external device 24 may include a touch screen display, and a user may interact with external device 24 via the display. It should be noted that the user may also interact with external device 24, server 26 and/or IMD 16 remotely via a networked computing device.

A user, such as a physician, technician, surgeon, electrophysiologist, or other clinician, may also interact with external device 24 or another separate programmer (not shown), such as a clinician programmer, to communicate with IMD 16 and/or server 26. Such a user may interact with external device 24 to retrieve physiological or diagnostic information from IMD 16. The user may also interact with external device 24 to program IMD 16, e.g., select values for the stimulation parameter values with which IMD 16 generates and delivers stimulation and/or the other operational parameters of IMD 16, such as magnitudes of stimulation energy, user requested periods for stimulation or periods to prevent stimulation, or any other such user customization of therapy. In some examples, the stimulation parameter values may be proposed by system 10, for example, by server 26 and a user may be able to accept or reject the stimulation parameter values. In other examples, the stimulation parameter values may be set by system 10, for example, by server 26. As discussed herein, the user may also provide input to external device 24 indicative of physiological events such as bladder fill level perception and void or leak events.

In some examples, a healthcare provider may utilize sensor 15, such as wearable sensors or existing implanted sensors, to collect patient data related to sleep, activity or disease symptoms. Sensor 15 may include one or more sensors, e.g., sensor(s) 15. For example, sensors 15 may be a heartrate sensor, an accelerometer and/or other sensor to collect patient data, for example, on disease symptoms or lifestyle. The patient data captured by the sensors, such as sensor 15, may be provided to server 26. In some examples, the sensors, such as sensor 15, may be configured to communicate with an external device, such as external device 24, via a wireless link. Additionally, sensors 15 may be any sensor that may generate one or more accompanying signals, e.g., accompanying signals which may indicate a patient posture at or near the time of delivery of electrical stimulation and/or sensing of stimulation-evoked signals (e.g., any or all of direct, indirect, single, and/or composite stimulation-evoked signals). In some examples, external device 24 may collect the patient data generated by the sensors and send the patient data to server 26. In other examples, another device may collect the patient data generated by the sensors and send the patient data to server 26.

In some examples, IMD 16 and/or external device 24 may receive information from sensor 15 directly, e.g., via wireless communication, or indirectly, such as from server 26 via a network connection. Sensor 15 may be positioned to sense one or more physiological responses at a selected location on patient 14. In some examples, sensor 15 may be positioned at, attached to or near tissue for a target anatomical area, e.g., at a limb or appendage, such as at or on a leg, toe, foot, arm, finger or hand of patient 14, e.g., to sense an EMG, a galvanic skin response adjacent to placement of sensor 15, or other response. In some examples, sensor 15 may be attached to an appendage of the patient 14 to sense a physiological response associated with the appendage, e.g., by a clip-on mechanism, strap, elastic band and/or adhesive. In some examples, sensor 15 (or one of a plurality of sensors 15) may be implantable within patient 14, e.g., within a limb or appendage of the patient, near the spinal cord of the patient, within the brain of the patient, and the like.

In some examples, sensor 15 may be a physiological and/or patient posture or behavior sensor, and may or may not be included with or within IMD 16. For example, sensor 15 may be a heart rate monitor configured to detect and/or determine a heart rate and/or a heart rate variability. Sensor 15 may be configured to detect and/or determine a biopotential. Sensor 15 may be a thermometer configured to detect and/or determine a temperature of at least a part of the patient's anatomy. Sensor 15 may be configured to measure a pressure, e.g., a patient blood pressure, or to measure an impedance of at least a portion of the patient's anatomy. Sensor 15 may be a blood flow sensor that measures blood flow and provides information related to blood flow associated with tissue of the patient. For example, sensor 15 may provide blood flow values, or other information indicative of blood flow values or changes in blood flow values. The blood flow value may be an instantaneous blood flow measurement or may be a measurement of blood flow over a period of time such as average blood flow value, maximum blood flow value, minimum blood flow value during the period of time. In some examples, sensor 15 may be a microphone configured to detect/determine sounds of at least a portion of the patient's anatomy. In some examples, sensor 15 may comprise and accelerometer configured to detect and/or determine a position and/or patient movement, a patient movement history over a predetermined amount of time, and the like. In some examples, sensor 15 may be configured to receive patient 14 input such as a pain response, a pain score, an area of pain, an amount of paresthesia, an area of paresthesia, information relating to voiding and/or a voiding rate (e.g., voids per day), and the like. In some examples, sensor 15 may be an environmental sensor, such as a microphone, thermometer, hygrometer, pressure sensor, and the like, configured to detect and/or determine sounds, temperatures, humidity and pressure, etc., of the environment in which the patient 14 is located.

The patient data and/or accompanying signals generated by the sensors, such as sensor 15, may be provided to server 26. In some examples, the sensors, such as sensor 15, may be configured to communicate with an external device, such as external device 24, via a wireless link. In some examples, external device 24 may collect the patient data and/or accompanying signals generated by the sensors and send the patient data to server 26. In other examples, another device may collect the patient data generated by the sensors and send the patient data and/or accompanying signals to server 26. In some examples, IMD 16 and/or external device 24 may receive information from sensor 15 directly, e.g., via wireless communication, or indirectly, such as from server 26 via a network connection.

In some examples, the user may use external device 24 to retrieve information from IMD 16 relating to the contraction frequency of bladder 12, frequency of voiding events, and/or a volume of fluid retained in one or more voiding events. As another example, the user may use external device 24 to retrieve information from IMD 16 relating to the performance or integrity of IMD 16 or other components of system 10, such as leads 18, 20, and 28, electrodes 29, 21, and 29, or a power source of IMD 16. In some examples, this information may be presented to the user as an alert.

The user of external device 24 may also communicate with server 26. For example, the user of external device 24 may provide information relating to the patient to server 26, such as demographic information, medical history, lifestyle information, bladder events, level satisfaction with therapy or sensor data.

Patient 14 may, for example, use a keypad or touch screen of external device 24 to request IMD 16 to deliver or terminate the electrical stimulation, such as when patient 14 senses that a leaking episode may be imminent or when an upcoming void may benefit from terminating therapy that promotes urine retention. In this way, patient 14 may use external device 24 to provide a therapy request to control the delivery of the electrical stimulation “on demand,” e.g., when patient 14 deems the second stimulation therapy desirable. This request may be a therapy trigger event used to terminate electrical stimulation. Patient 14 may also use external device 24 to provide other information to IMD 16, such as information indicative of a phase of a physiological cycle, such as the occurrence of a voiding event.

External device 24 may provide a notification to patient 14 when the electrical stimulation is being delivered or notify patient 14 of the prospective termination of the electrical stimulation. In addition, notification of termination may be helpful so that patient 14 knows that a voiding event may be more probable and/or the end of the fill cycle is nearing such that the bladder should be emptied (e.g., the patient should visit a restroom). In such examples, external device 24 may display a visible message, emit an audible alert signal or provide a somatosensory alert (e.g., by causing a housing of external device 24 to vibrate). In other examples, the notification may indicate when therapy is available (e.g., a countdown in minutes, or indication that therapy is ready) during the physiological cycle. In this manner, external device 24 may wait for input from patient 14 prior to terminating the electrical stimulation that reduces bladder contraction or otherwise promotes urine retention. Patient 14 may enter input that either confirms termination of the electrical stimulation so that the therapy stops for voiding purposes, confirms that the system should maintain therapy delivery until patient 14 may void, and/or confirms that patient 14 is ready for another different stimulation therapy that promotes voiding during the voiding event.

In the event that no input is received within a particular range of time when a voiding event is predicted, external device 24 may wirelessly transmit a signal that indicates the absence of patient input to IMD 16. IMD 16 may then elect to continue stimulation until the patient input is received, or terminate stimulation, based on the programming of IMD 16. In some examples, the termination or continuation of electrical stimulation may be responsive to other physiological markers.

IMD 16 and external device 24 may communicate via wireless communication using any techniques known in the art. Examples of communication techniques may include, for example, Bluetooth®, low frequency or radiofrequency (RF) telemetry, but other techniques are also contemplated. In some examples, external device 24 may include a programming lead that may be placed proximate to the patient's body near the IMD 16 implant site in order to improve the quality or security of communication between IMD 16 and external device 24.

IMD 16, in response to commands from external device 24, may deliver electrical stimulation therapy according to a one or more stimulation programs to a target tissue site of the patient 14 via any of electrodes 29A-29D, 19A-19B, and 21A-21B. In some examples, IMD 16 automatically modifies therapy stimulation programs as therapy needs of patient 14 evolve over time. For example, the modification of the therapy stimulation programs may cause the adjustment of at least one parameter of the plurality of stimulation pulses based on received information.

In the example four-wire arrangement shown in FIG. 1, electrodes 19A and 21A and electrodes 19B and 21B, may be located substantially opposite each other relative to the center of bladder 12. For example, electrodes 19A and 21A may be placed on opposing sides of bladder 12, either anterior and posterior or left and right. In FIG. 1, electrodes 19 and 21 are shown placed proximate to an exterior surface of the wall of bladder 12. In some examples, electrodes 19 and 21 may be sutured or otherwise affixed to the bladder wall. In other examples, electrodes 19 and 21 may be implanted within the bladder wall. To measure the impedance of bladder 12, IMD 16 may source an electrical signal, such as current, to electrode 19A via lead 18, while electrode 21A via lead 20 sinks the electrical signal. IMD 16 may then determine the voltage between electrode 19B and electrode 21B via leads 18 and 20, respectively. IMD 16 determines the impedance of bladder 12 using a known value of the electrical signal sourced the determined voltage.

In other examples, electrodes 19, 21 and 29 may be used to detect an electromyogram (EMG) of the detrusor muscle. This EMG may be used to determine the frequency of bladder contractions and the physiological marker of patient 14. The EMG may also be used to detect the strength of the bladder contractions, or to determine a bladder or bowel emptying, in some examples. As an alternative, or in addition, to an EMG, a strain gauge or other device may be used to detect the status of bladder 12, e.g., by sensing forces indicative of bladder contractions.

In the example of FIG. 1, IMD 16 also may include a sensor 22 for sensing one or more accompanying signals, such as detecting changes in the contraction of bladder 12. Sensor 22 may include, for example, a pressure sensor for detecting changes in bladder pressure. In some examples, in lieu of or addition to sensor 22, IMD 16 may also include additional sensors such as electrodes (which may be any of electrodes 19, 21, or 29, but may also be additional electrodes not shown) that may be coupled to or placed near bladder 12, e.g., for sensing pudendal or sacral nerve signals (e.g., afferent and/or efferent), electrodes for sensing urinary sphincter EMG signals (or anal sphincter EMG signals in examples in which system 10 provides therapy to manage fecal urgency or fecal incontinence), or any combination thereof In examples in which sensor 22 is a pressure sensor, the pressure sensor may be a remote sensor that wirelessly transmits signals to IMD 16 or may be carried on one of leads 18, 20, or 28 or an additional lead coupled to IMD 16. In some examples, IMD 16 may determine whether a contraction of bladder 12, e.g., related to a void, detrusor muscle activity, bladder state change such as size or shape or any other change, has occurred based on a pressure signal generated by sensor 22, a signal generated by a volume sensor indicating fullness or emptiness, or the like. In some examples, IMD 16 may control the timing of the delivery of the electrical stimulation based on input received from sensor 22.

In some examples, sensor 22 may sense one or more stimulation-evoked signals. For example, stimulation-evoked signals may include signals at the site of electrical stimulation delivery (local/proximal to the stimulation electrode/nerve interface and/or also distributed stimulation-evoked signals arising from distal locations that are related to the original evoked response via neural, physiologic and/or anatomic mechanisms). Sensor 22 may sense stimulation-evoked signals distally from the site of stimulation delivery and/or arising from propagation/translation of the initial evoked signal/activation.

In examples in which IMD 16 includes one or more electrodes for sensing afferent nerve signals, the sense electrodes may be carried on one of leads 18, 20, or 28 or an additional lead coupled to IMB 16. In examples in which IMB 16 includes one or more sense electrodes for generating a urinary sphincter EMG, the sense electrodes may be carried on one of leads 18, 20, or 28 or additional leads coupled to IMB 16. In any case, in some examples, IMD 16 may control the timing of the delivery of the electrical stimulation based on input received from sensor 22.

Sensor 22 may comprise a patient motion sensor that generates an accompanying signal indicative of a patient posture, such as an activity level or posture state. In some examples, IMD 16 may terminate the delivery of the electrical stimulation to patient 14 upon detecting a patient activity level exceeding a particular threshold based on the signal from the motion sensor. In other examples, IMB 16 may use sensor 22 to identify patient posture states known to require the desired therapeutic effect. For example, patient 14 may be more prone to an involuntary voiding event when patient 14 is in an upright posture state compared to a lying down posture state. In some examples, electrodes 19 and 21 and sensor 22 may be configured to detect voiding events and/or the magnitude of a fill level of bladder 12 during the fill cycle. In some examples, IMD 16 may include sensor 22 and/or a motion sensor, e.g., within the housing of IMD 16.

As discussed above, system 10 may monitor the fill cycle of bladder 12 by detecting subsequent voiding events over time. In some examples, system 10 may detect voiding events via electrodes 19, 21 and 29, e.g., via a signal received by electrodes 19, 21 and 29. In some examples, system 10 may detect voiding events by receiving an indication of a user input (e.g., via external device 24) representative of an occurrence of a voiding event. In other words, external device 24 may receive input from the user identifying that a voiding event occurred, the beginning of a voiding event, and/or the end of the voiding event. In other examples, system 10 may automatically detect voiding events without receiving user input via external device 24. System 10 may instead detect voiding events by detecting at least one of a pressure of the bladder, a flow of urine from the bladder, a wetness of an article external of the patient, a volume of the bladder, an EMG signal, a nerve recording, a posture change, a physical location of the patient within a structure such as a house or care facility, or a toilet use event. Some sensors external to patient 14 may communicate with external device 24 and/or IMD 16 to provide this information indicative of likely voiding events. For example, wetness may be detected by a moisture sensor (e.g., electrical impedance or chemical sensor) embedded in an undergarment worn by the patient and transmitted to IMD 16 or external device 24. Similarly, a toilet may include a presence sensor that detects when a patient is using the toilet (e.g., an infrared sensor, thermal sensor, or pressure sensor) and transmits a signal indicating the presence of the patient to IMD 16 or external device 24. In this manner, non-invasively obtained data may provide information indicative of voiding events without implanted sensors. The information indicative of voiding events may be provided to server 26 by external device 24 or IMD 16. System 10 of FIG. 1 may implement the techniques of this disclosure.

In accordance with one or more aspects of this disclosure, IMD 16, external device 24, server 26, or another device may determine a quality of stimulation-evoked signals sensed by electrodes 19, 21, and 29. In some examples, a quality of stimulation-evoked signals may be a reliability and/or a trustworthiness of stimulation-evoked signals, e.g., whether and to what degree the sensed stimulation-evoked signals accurately indicate responses of nerves and/or muscles (e.g., signal sources) to electrical stimulation signals delivered by an electrical stimulation device. In some examples, the quality may be a qualitative or a quantitative measure. For example, the quality may be a numerical rating on a scale, such as a particular value from a minimum to maximum, (e.g., a 3 on a scale of 1 to 10 indicating where 1 is a minimum quality and 10 is a maximum quality). The quality may be qualitative level on a scale, e.g., a high quality on a low-medium-high quality scale. In some examples, the quality may be a determined and/or calculated numerical value.

For example, IMD 16 and/or external device 24 may execute a procedure to determine the efficacy of an electrical stimulation therapy program by delivering electrical stimulation signals according to the therapy program, sensing and capturing stimulation-evoked signals from one or more signal source (e.g., nerves and/or muscles) indicative of the response of the signal source to the delivered electrical stimulation and optionally sensing and capturing one or more accompanying signals, and processing the stimulation-evoked signals and/or accompanying signals to determine responses of the signal sources and efficacy of the delivered electrical stimulation signals.

In some examples, before making determinations and deriving information from the captured stimulation-evoked signals, IMD 16 and/or external device 24 may first determine whether the captured stimulation-evoked signals are good enough, e.g., are of high enough quality, so as to make a determination regarding the uncertainty, reliability, and/or trustworthiness of determinations and/or information derived from and/or based on the captured stimulation-evoked signals. By way of a nonlimiting example, IMD 16 and/or external device 24 may determine that patient 14 was moving and/or active during delivery of the electrical stimulation and/or sensing of stimulation-evoked signals, e.g., based on sensor 15 and/or sensor 22 accelerometer data. IMD 16 and/or external device 24 may then determine that the quality of the sensed and captured stimulation-evoked signals may be too low, e.g., below a quality threshold, because it may be known, for that particular electrical stimulation therapy program, that the patient 14 should not be moving during sensing or the stimulation-evoked signals will be too noisy, contain too many artifacts (e.g., spurious muscle contractions that are not in response to the delivered electrical stimulation), and/or otherwise not be accurately indicative of responses of signal sources to the delivered electrical stimulation signals.

In some examples, IMD 16 and/or external device 24 (or server 26, or another device) may output the determined quality, and may further output instructions to improve the quality of stimulation-evoked signals that are to be subsequently sensed based on the determined quality being below a quality threshold. That is, IMD 16 and/or external device 24 may determine that the quality of stimulation-evoked signals is not good enough to derive further information and/or make determinations based on the stimulation-evoked signals and may provide instructions, e.g., to patient 14, a clinician, a user of system 10 and/or a device such as IMD 16, to take an action to improve the quality of subsequent stimulation-evoked signals. For example, external device 24 may provide instructions to patient 14 via a display of external device 24 to reduce movement, sit, or lie down for a period of time for another electrical stimulation therapy session and measurement. In another example, external device 24 may provide instructions to IMD 16 to change the therapy program, e.g., change one or more parameters of the electrical stimulation, for another electrical stimulation therapy session and measurement.

For example, IMD 16 and/or external device 24 may be configured to cause one or more electrodes 19, 21, and 29 to deliver one or more electrical stimulation signals to patient 14. In some examples, IMD 16 and/or external device 24 may cause one or more electrodes 19, 21, and 29 to deliver one or more electrical stimulation signals having non-equal pulse amplitudes, non-equal pulse durations, non-equal polarities, and/or non-equal pulse frequencies. In some examples, IMD 16 and/or external device 24 may cause one or more electrodes 19, 21, and 29 to deliver a plurality of electrical stimulation signals according to a known and/or predetermined progression of parameters, e.g., in a “sweep” such as an amplitude or frequency sweep, or the like, alone or in any combination. In some examples, IMD 16 and/or external device may be configured to deliver one or more electrical stimulation signals to a sacral nerve (e.g., for SNM therapy), a peripheral nerve (e.g., for PNS and/or PNFS), a saphenous nerve, a tibial nerve, a pudendal nerve, a sciatic nerve, or any other suitable nerve, muscle, and or tissue of patient 14.

In some examples, one or more signal sources, such as one or more nerves, one or more muscles, or at least one muscle and at least one nerve, may respond to the electrical stimulation, e.g., via a neural response, a muscle contraction and/or activation, or any other response. In some examples, the response of the one or more sources may be electrical, e.g., an EMG, an ECAP or surface EMG, and the like. In some examples, the response may be mechanical and converted to an electrical signal by a sensor or detector, e.g., by a piezoresistive sensor or other sensor configured to measure muscle contraction and mechanomyography (MMG) and the like. In some examples, nerves may include any of the sacral nerves, dorsal and ventral rami of sacral nerves, pudendal nerves, sciatic nerves, saphenous nerves, nerves in the sacral plexus, pelvic nerves, pelvic plexus nerves, pelvic splanchnic nerves, inferior hypogastric plexus nerves, lumbosacral trunk nerves, e.g., where the lumbosacral trunk joins sacral nerves, any sympathetic nerve fibers in the sympathetic chain of any of the above nerves or other nerves. In some examples, one or more muscles may include an external anal sphincter muscle, coccygeus muscle, levator ani muscle group, bulbocavernosus and/or bulbospongiosus muscle, gluteal muscles, e.g., gluteal maximus, gluteal medius, and gluteal minimus, perineal muscles, ischiocavernosus muscles, puborectalis muscles, piriformis muscles, erector spinae muscles, multifidus muscle, rectum, or any other muscles.

In some examples, the stimulation-evoked signals may comprise a composite stimulation-evoked signal comprising a composite of signals generated by one or more signal sources in response to the one or more electrical stimulation signals via a neural response, a muscle contraction and/or activation, or any other response. In some examples, the composite stimulation-evoked signal sensed by one or more electrodes and may be a combination of any and/or all of the various signal sources. For example, an electrical stimulation signal may cause a nerve and/or muscle proximate to the stimulation signal to generate a response and other nerves or muscles, not necessarily proximate to the stimulation signal, may also generate responses. The composite stimulation-evoked signal may be a composite of signals from any of the multiple signal sources.

One or more sensors and/or electrodes, such as sensors 15, sensor 22, and/or electrodes 19, 21, and 29, may receive and/or sense signals from the one or more signal sources. In some examples, the received signals may be a composite of sensed signals from one or more sensors 15, sensor 22, and/or electrodes 19, 21, and 29. In other examples, the received signals may be a composite of sensed signals from just electrodes, e.g., one or more electrodes 19, 21, and 29. For example, electrodes 19, 21, and/or 29 may receive and/or sense the signals from one or more signal sources concurrently over a period of time as a single composite stimulation-evoked signal. For example, two or more signals may “arrive” at the sensor (or sensors or electrodes) at the same time and may add together forming the composite signal that is sensed. For example, the two or more signals may be electric signals which may add incoherently, coherently, constructively, destructively, and the like, to form the electric signal that is sensed. In other examples, the two or more signals may be individually sensed and then added and/or combined to for the composite stimulation-evoked signal. For example, electrodes 29 may sense an electric field caused by neural activity of nerve and a sensor 15 may sense an EMG signal caused by a contraction of a muscle, both in response to delivered electrical stimulation, and which may be combined forming a composite stimulation-evoked signal. IMD 16 and/or external device 24 may receive each stimulation-evoked signal from two or more sources and then combine the signals to form the composite stimulation-evoked signal.

In some examples, one or more electrodes, such as electrodes 29, may receive and/or sense signals from the one or more signal sources, e.g., electrodes 29 may sense one or more stimulation-evoked signals, or a composite stimulation-evoked signal. For example, the received signals may be a composite, e.g., electrodes 29, may receive and/or sense the signals from one or more signal sources concurrently over a period of time as a single composite stimulation-evoked signal. For example, two or more signals may “arrive” at electrodes 29 at the same time and may add together forming the composite signal that is sensed. For example, the two or more signals may be electric signals which may add incoherently, coherently, constructively, destructively, and the like, to form the electric signal that is sensed. In other examples, the two or more signals may be individually sensed and then added and/or combined to for the composite stimulation-evoked signal. For example, electrodes 29 may sense an electric field caused by neural activity of nerve and may sense an EMG signal caused by a contraction of a muscle, both in response to delivered electric stimulation. IMD 16 and/or external device 24 may receive each stimulation-evoked signal from each of the one or more sources and then combine the signals to form the composite stimulation-evoked signal.

In some examples, the one or more signal sources may be located relatively far from one or more sensing electrodes, e.g., a sensor/electrode (e.g., sensors 15, sensor 22, and/or electrodes 19, 21, and 29) and/or each other, e.g., at least 5 millimeters (mm) from the sensor and/or electrode and/or each other, at least 10 mm from the sensor and/or electrode and/or each other, at least 100 mm from the sensor and/or electrode and/or each other, at least 200 mm from the sensor and/or electrode and/or each other, at least 1 meter from the sensor and/or electrode and/or each other. For example, one or more signal sources may include a tibial nerve responding to sacral nerve stimulation.

In some examples, the composite stimulation-evoked signal may have a relative long duration, e.g., more than 1 millisecond (ms), more than 3 ms, more than 5 ms, more than 10 ms, more than 20 ms, etc. For example, because the composite stimulation-evoked signal may originate from multiple signal sources at multiple distances from one or more the sensors and/or electrodes, and because different signal sources may have different response times, the signals from the signal sources may arrive at, and be captured by, a sensor and/or electrode at different times. In some examples, a sensor and/or electrode may sense signals from signal sources after delivery of every electrical stimulation signal, or a sensor and/or electrode may sense signals from signal sources after an amount of time after delivery of electrical stimulation signals.

In some examples, the one or more signal sources may be located relatively close to one or more sensing electrodes, e.g., a sensor/electrode (e.g., sensors 15, sensor 22, and/or electrodes 19, 21, and 29) and/or each other, e.g., within 100 mm from the sensor and/or electrode and/or each other, within 50 mm from the sensor and/or electrode and/or each other, within 20 mm from the sensor and/or electrode and/or each other, within 10 mm from the sensor and/or electrode and/or each other, within 5 mm from the sensor and/or electrode and/or each other.

In some examples, the composite stimulation-evoked signal may have a relative short duration, e.g., less than 10 ms, less than 5 ms, less than 3 ms, less than 1 ms, less than 0.5 ms, etc. For example, because the composite stimulation-evoked signal may comprise ECAP signals.

In some examples, the composite stimulation-evoked signal may comprise signals of different types from different signal sources. For example, the composite stimulation-evoked signal may comprise an ECAP signal generated relatively quickly after delivery of electrical stimulation signals, e.g., within about 5 ms, and an EMG signal generated relatively slowly after delivery of electrical stimulation signals, e.g., after about 5 ms. In some examples, the composite stimulation-evoked signal may comprise signals from the same signal source or multiple signal sources that do not overlap in time. For example, the composite stimulation-evoked signal may comprise an ECAP signal from a signal source relative close to the sensor and/or electrode followed by an EMG signal or another ECAP signal from a different signal source, e.g., after a reflex in the spinal cord, that may be relatively far from the sensor and/or electrode, e.g., such that the ECAP from the close signal source is no longer present while the EMG signal and/or ECAP from the more distant signal source are received by the sensor and/or electrode. In some examples, the composite stimulation-evoked signal may comprise two or more EMG signals, e.g., from one or more signal sources. For example, the composite stimulation-evoked signal may comprise a first EMG signal generated relatively quickly after delivery of electrical stimulation signals, e.g., within about 5 ms, or within about 0.5 ms, and a second EMG signal generated relatively slowly after delivery of electrical stimulation signals, e.g., after about 5 ms.

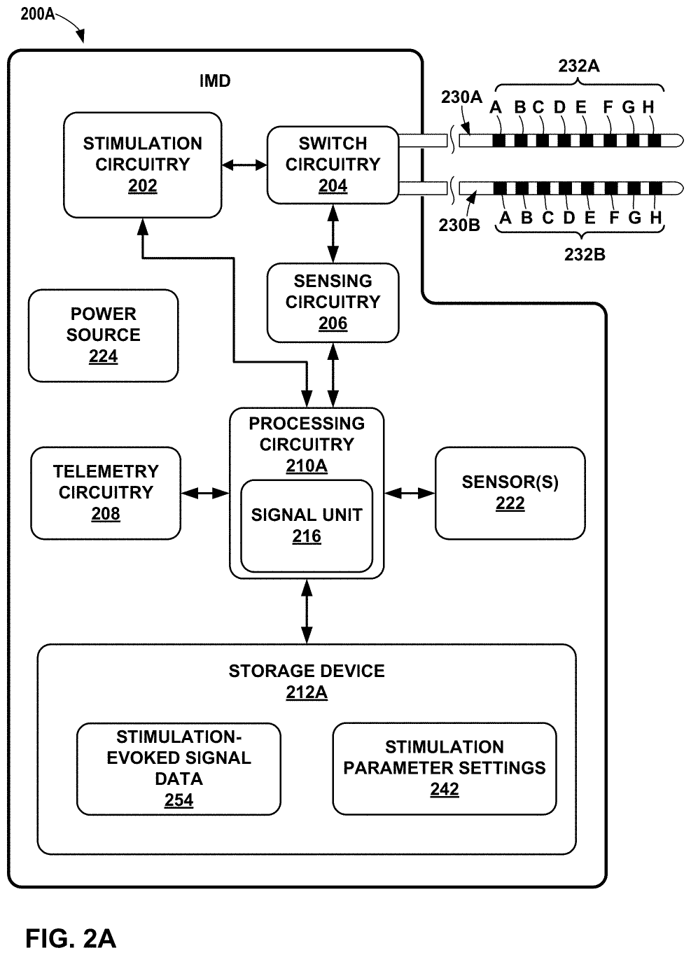

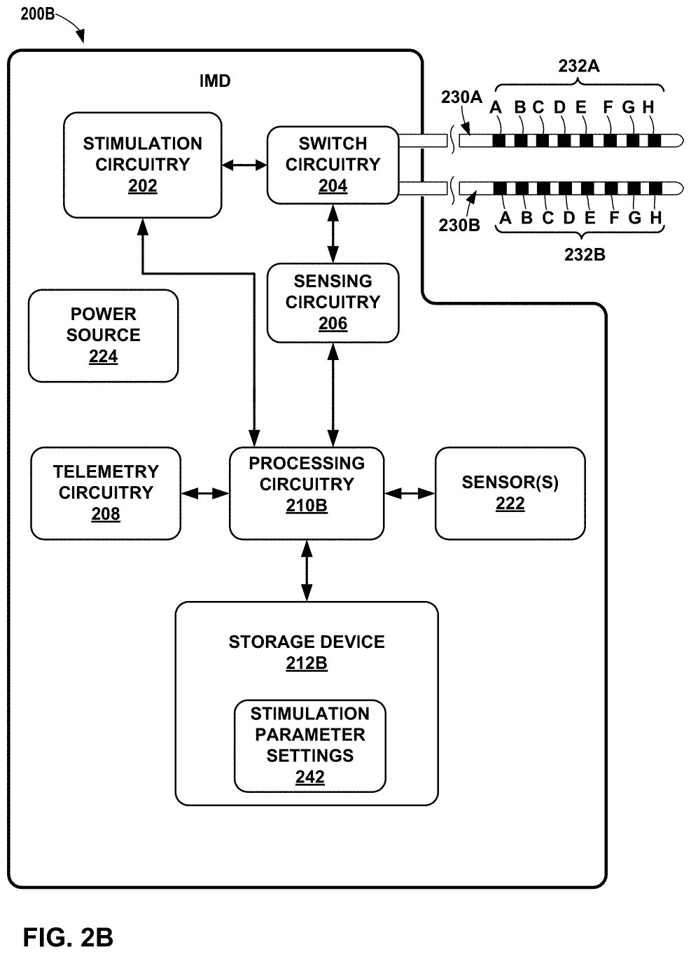

FIG. 2A and 2B are block diagrams illustrating example configurations of components of an IMD 200A and an IMD 200B, respectively, in accordance with one or more techniques of this disclosure. IMD 200A and/or IMD 200B may be an example of IMD 16 of FIG. 1, or of an external device configured to perform the functionality of IMD 16. In the examples shown in FIGS. 2A and 2B, IMD 200A and IMD 200B each include stimulation generation circuitry 202, switch circuitry 204, sensing circuitry 206, telemetry circuitry 208, sensor(s) 222, power source 224, lead 230A carrying electrodes 232A, which may correspond to one of leads 18, 20, 28 and electrodes 19, 21, 29 of FIG. 1, and lead 230B carrying electrodes 232B, which may correspond to another one of leads 18, 20, 28 and electrodes 19, 21, 29 of FIG. 1. In the examples shown in FIG. 2A, IMD 200A includes processing circuitry 210A and storage device 212A, and in the example shown in FIG. 2B, IMD 200B includes processing circuitry 210B and storage device 212B. Processing circuitry 210A and/or 210B may include one or more processors configured to perform various operations of IMD 200A and/or IMD 200B.

In the examples shown in FIGS. 2A and 2B, storage devices 212A and 212B store stimulation parameter settings 242. In addition, as shown in FIG. 2A, storage device 212A may store stimulation-evoked signal data 254 obtained directly or indirectly from one or more electrodes 232, e.g., via sensing circuitry 206, and/or sensors 222, e.g., which may correspond to any or all of electrodes 19, 21, 29 and/or sensors 15, 22 (FIG. 1). In this case, IMD 200A of FIG. 2A may process stimulation-evoked signal data 254 and select or adjust stimulation parameter settings 242, including cycling, based on the stimulation-evoked signal data 254.

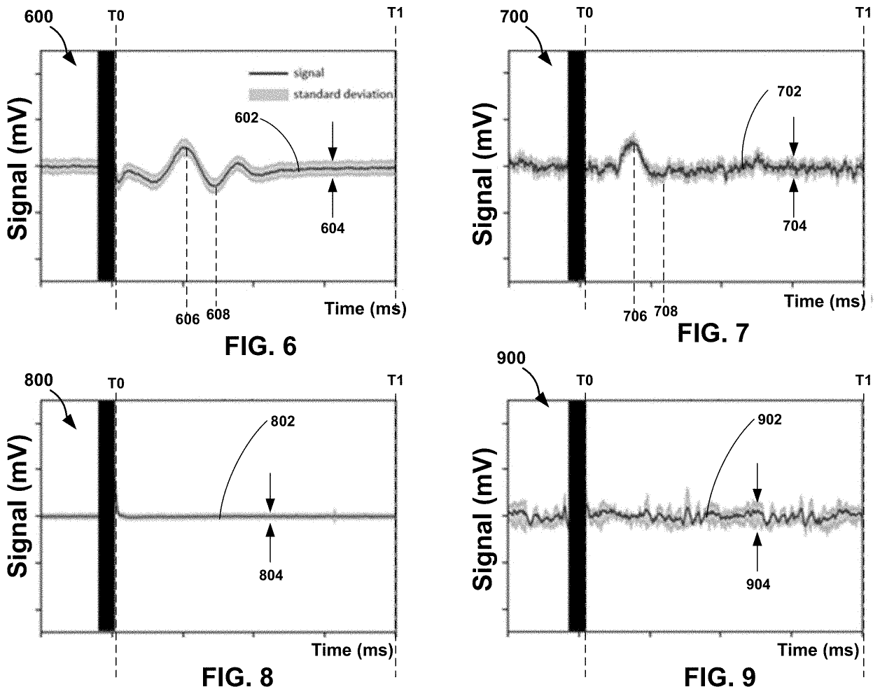

Stimulation-evoked signal data 254 may include sensed stimulation-evoked signals generated by one or more signal sources (e.g., which may be stimulation-evoked and referred to as stimulation-evoked signals), sensed composite stimulation-evoked signals, such as those described above, and/or accompanying signals, e.g., which may be indicative of a quality of sensed stimulation-evoked signals. In some examples, stimulation-evoked signal data 254 may include raw sensed signals from sensor(s) 222 and/or electrodes 232A, 232B and/or amplified, filtered, averaged, and/or analog-to-digital converted signals, e.g., via sensing circuitry 206. For example, stimulation-evoked signal data 254 may include a time-varying signal indicative of a response or responses of one or more signal sources (e.g., nerves and/or muscles) to electrical stimulation, such as illustrated and described below with reference to FIGS. 6-19. In some examples, stimulation-evoked signal data 254 may include an averaged signal and/or one or more signal features determined via processing of the signal, e.g., peak/valley detection, peak/valley amplitude, width, and/or area, frequency analysis, digital signal processing, signal latency, and the like, or any ratios and/or combinations thereof. In some examples, stimulation-evoked signal data 254 may include additional information, such as sensor(s) 222 settings during sensing of stimulation-evoked signals, a timestamp denoting the date and/or time one or more stimulation-evoked signals are sensed, patient information including a current physiological state of patient 14 and/or physiological measurements of patient 14 at or near the time one or more stimulation-evoked signals are sensed, e.g., heart rate, temperature, blood pressure, patient activity, motion, and/or posture (e.g., patient input and/or measured, such as from a patient smartphone, wearable device, external device 24 or 300, or other device) and the like, or patient input such as a pain level and/or pain score, voiding and/or voiding frequency, post-void volume, a number and/or frequency of catheterizations, patient medical history information, patient age or other demographic information, or any other suitable patient input information.

In one or more examples, such as shown in FIG. 2B, the IMD 200B may not store or receive the stimulation-evoked signal data 254. Instead, external device 24 or another device may directly or indirectly select or adjust stimulation parameter settings based on stimulation-evoked signal data 254 and communicate the selected settings or adjustments to IMD 200B of FIG. 2B. In some examples, stimulation parameter settings 242 may include stimulation parameters (sometimes referred to as “sets of therapy stimulation parameters”) for respective different stimulation programs selectable by the clinician or patient for therapy. For example, stimulation parameter settings 242 may include, but may not be limited to, electrode combination or configuration, electrode polarity, amplitude, pulse width, pulse shape, pulse frequency or pulse rate, or cycling. In some examples, stimulation parameter settings 242 may include one or more recommended parameter settings. In this manner, each stored therapy stimulation program, or set of stimulation parameters, of stimulation parameter settings 242 defines values for a set of electrical stimulation parameters (e.g., a stimulation parameter set), such as electrode combination (selected electrodes and polarities), stimulation current or voltage amplitude, stimulation pulse width, and pulse frequency.

In some examples, stimulation parameter settings 242 may indicate for the stimulation to turn on for a certain period of time, and/or to turn off stimulation for a certain period of time. For example, stimulation parameter settings 242 may further include cycling information indicating when or how long stimulation is turned on and off, e.g., periodically and/or according to a schedule. For example, electrical stimulation may be delivered as a series of electrical stimulation pulses, each pulse being defined by an amplitude, a frequency, a pulse width and/or duration, and an electrode combination (e.g., stimulation pulse parameters). Cycling parameters may define how the series of pulses is delivered. For example, stimulation cycling parameters may include a cycling frequency or period and a duty cycle or ratio of how long electrical stimulation pulses are delivered according to the cycling frequency (an “on-time”) to how long electrical stimulation is not delivered (an “off-time). In other examples, cycling may include a schedule defining the specific times at which electrical stimulation pulses are to be delivered according to specific stimulation pulse parameter settings.

In some examples, cycling and/or a schedule may include variation over time of any of the electrode combination, amplitude, pulse frequency, pulse width, cycling frequency, and cycling duty cycle, such as a taper in which a parameter is decreased and/or increased. As one specific example of just two parameters, a cycling parameter may include a constant or variable rate of decrease of the amplitude of the pulses and the duty cycle (e.g., a decrease in the on-time/off-time ratio). In some examples, stimulation parameter settings 242 may further include other information and/or limits to other stimulation parameter settings, e.g., such as stimulation pulse or cycling parameter settings limits to deliver electrical stimulation therapy without creating, or to reduce, desensitization of the patient to the electrical stimulation. In some examples, stimulation parameter settings 242 may indicate when stimulation is to occur, e.g., periodically, according to a predetermined schedule, at a certain time of day, for example when the patient is typically awake or active, or sleeping, based on patient posture, or the like. In some examples, stimulation parameter settings 242 relate to when the patient has a certain posture, for example only deliver stimulation when the patient is in a supine position.

In some examples, an electrical stimulation signal may comprise electrical stimulation delivered according to one or more electrical stimulation parameter settings 242, e.g., electrical stimulation delivered according to stimulation pulse parameters settings, stimulation cycling parameters settings, and/or any other suitable stimulation parameters settings, information, limits, or conditions.

Stimulation generation circuitry 202 includes electrical stimulation circuitry configured to generate electrical stimulation and generates electrical stimulation pulses selected to alleviate symptoms of one or more diseases, disorders or syndromes. While stimulation pulses are described, stimulation signals may take other forms, such as continuous-time signals (e.g., sine waves) or the like. The electrical stimulation circuitry may reside in an implantable housing, for example of the IMD. In other examples, the electrical stimulation circuitry may reside in an external medical device housing (not shown), e.g., an external device including the circuitry and functionality of IMD200A and/or IMD200B described herein and configured to directly connect to leads 230A, 230B. Each of leads 230A, 230B may include any number of electrodes 232A, 232B. The electrodes are configured to deliver the electrical stimulation to the patient. In the example of FIGS. 2A and 2B, each set of electrodes 232A, 232B includes eight electrodes A-H. Although electrode sets 232A, 232B are described with eight electrodes, electrode sets 232A, 232B may have more or fewer electrodes, for example, electrode sets 232A, 232B may have a single electrode, two electrodes, three electrodes, four electrodes, or five, six, or seven electrodes, or nine or more electrodes. In the example shown, electrode sets 232A and 232B have the same number of electrodes. In other examples, electrode sets 232A and 232B may have a different number of electrodes from each other. In some examples, the electrodes are arranged in bipolar combinations. A bipolar electrode combination may use electrodes carried by the same lead 230A, 230B or different leads. For example, an electrode A of electrodes 232A may be a cathode and an electrode B of electrodes 232A may be an anode, forming a bipolar combination. In other examples, the electrodes may be monopolar. For example, a housing of lead 230A and/or lead 230B, or IMD 16, or a ground patch (not shown) may function as the return path for one or more electrodes 232A and/or electrodes 232B in a monopolar configuration. Switch circuitry 204 may include one or more switch arrays, one or more multiplexers, one or more switches (e.g., a switch matrix or other collection of switches), or other electrical circuitry configured to direct stimulation signals from stimulation generation circuitry 202 to one or more of electrodes 232A, 232B, or directed sensed signals from one or more of electrodes 232A, 232B to sensing circuitry 206. In some examples, one or more of electrodes 232A, 232B may be configured to both deliver stimulation signals and sense signals, and switch circuitry 204 may be configured to direct stimulation signals from stimulation generation circuitry 202 to such electrodes and direct sensed signals, sensed by such electrodes, to sensing circuitry 206. In some examples, each of the electrodes 232A, 232B may be associated with respective regulated current source and sink circuitry to selectively and independently configure the electrode to be a regulated cathode or anode. Stimulation generation circuitry 202 and/or sensing circuitry 206 also may include sensing circuitry to direct electrical signals sensed at one or more of electrodes 232A, 232B.

Sensing circuitry 206 may be configured to monitor and/or process (e.g., amplify, filter, analog-to-digital convert, etc.) signals from any combination of electrodes 232A, 232B and/or sensor(s) 222. In some examples, sensing circuitry 206 includes one or more amplifiers, filters, and analog-to-digital converters. Sensing circuitry 206 may be used to sense stimulation-evoked and/or physiological signals, such as EMG signals, ECAP signals, and the like. In some examples, sensing circuitry 206 detects EMG, ECAP, and/or signals from a particular combination of electrodes 232A, 232B. In some cases, the particular combination of electrodes for sensing ECAP and/or EMG signals includes different electrodes than a set of electrodes 232A, 232B used to deliver stimulation pulses. Alternatively, in other cases, the particular combination of electrodes used for sensing ECAP, EMG, and/or EMG signals includes at least one of the same electrodes as a set of electrodes used to deliver stimulation pulses to patient 14. Sensing circuitry 206 may provide signals to an analog-to-digital converter, for conversion into a digital signal for processing, analysis, storage, or output by processing circuitry 210. In some examples, sensing circuitry 206 may sense and/or detect stimulation-evoked signals and/or composite stimulation-evoked signals comprising one or more of an ECAP, an EMG or surface EMG, an MMG, a network excitability, and/or multiple signals of differing signal type evoked by one or more signal sources such as sacral nerves, e.g., dorsal and ventral rami of sacral nerves, pudendal nerves, sciatic nerves, saphenous nerves, nerves in the sacral plexus, pelvic nerves, pelvic plexus nerves, pelvic splanchnic nerves, inferior hypogastric plexus nerves, lumbosacral trunk nerves, e.g., where the lumbosacral trunk joins sacral nerves, any sympathetic nerve fibers in the sympathetic chain of any of the above nerves or other nerves, muscles such as an external anal sphincter muscle, coccygeus muscle, levator ani muscle group, bulbocavernosus and/or bulbospongiosus muscle, gluteal muscles, e.g., gluteal maximus, gluteal medius, and gluteal minimus, perineal muscles, ischiocavernosus muscles, puborectalis muscles, piriformis muscles, or any other muscles.

Sensor(s) 222 may be substantially the same as sensors 15, 22 described above with reference to FIG. 1. In some examples, sensors 222 may be similar to sensors 15, 22 described above except that sensors 222 may be located within the housing of the IMD, e.g., IMD 16, IMD 200A, or IMD 200B. In some examples, sensors 222 may be configured to sense stimulation-evoked signals and/or accompanying signals. For example, sensor(s) 222 may be configured to sense and/or detect and/or capture one or more physiological responses of a patient, e.g., patient 14. In some examples, sensors 222 may be other sensors located at one or more other positions on patient 14, located at or near one or more muscles and or nerves, or located at positions on patient 14 which may be relatively far from a signal source, e.g., a nerve or muscle. In some examples, sensors 222 may be configured to sense some other type of quantity other than a stimulation-evoked signal that may be indicative of the quality of a stimulation-evoked signal, e.g., patient movement, heart rate data, pressure sensor data, and the like, and may generate one or more corresponding accompanying signals.

Telemetry circuitry 208 supports wireless communication between IMD 200A and/or IMD 200B and an external programmer or another computing device under the control of processing circuitry 210. Processing circuitry 210A and/or 210B of IMD 200A and/or IMD 200B, respectively, may receive, as updates to programs, values for various stimulation parameters such as amplitude and electrode combination, from the external programmer via telemetry circuitry 208. Processing circuitry 210A and/or 210B of IMD 200A and/or IMD 200B, respectively, may store updates to the stimulation parameter settings 242 or any other data in storage device 212. Telemetry circuitry 208 in IMD 200A and/or IMD 200B, as well as telemetry circuits in other devices and systems described herein, such as the external programmer and patient feedback sensing system, may accomplish communication by radiofrequency (RF) communication techniques. In addition, telemetry circuitry 208 may communicate with an external medical device programmer via proximal inductive interaction of IMD 200A and/or IMD 200B with the external programmer, where the external programmer may be one example of external device 24 of FIG. 1. Accordingly, telemetry circuitry 208 may send information to the external programmer on a continuous basis, at periodic intervals, or upon request from IMD 16 and/or external device 24.

Processing circuitry 210A and/or 210B may include one or more processors, such as any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210A and/or 210B herein may be embodied as firmware, hardware, software or any combination thereof. Processing circuitry 210A and/or 210B controls stimulation generation circuitry 202 to generate stimulation signals according to stimulation parameter settings 242. In some examples, processing circuitry 210A and/or 210B may execute other instructions stored in storage device 212A and/or 212B, respectively, to apply stimulation parameters specified by one or more of programs, such as electrode combination or configuration, electrode polarity, amplitude, pulse width, pulse shape, pulse frequency or pulse rate, cycling of each of the stimulation signals, or according to a known and/or predetermined progression of parameters, e.g., in a sweep such as an amplitude or frequency sweep, or the like, alone or in any combination. In some examples, processing circuitry 210A and/or 210B may execute other instructions stored in storage device 212A and/or 212B, respectively, to apply sensing parameters specified by one or more of programs, such as electrode combination or configuration, electrode polarity, according to a known and/or predetermined progression of sensing parameters, e.g., in a sweep of such parameters.

In the illustrated example of FIG. 2A, processing circuitry 210A includes a signal unit 216 to process stimulation-evoked signals (e.g., which herein may refer to stimulation-evoked signals from individual signal sources and/or composite stimulation-evoked signals including stimulation-evoked signals from one or more signal sources), and/or accompanying signals. Signal unit 216 may represent an example of a portion of processing circuitry configured to process stimulation-evoked signals and/or accompanying signals received from a sensor, such as any or all of electrodes 232A, 232B and/or sensor(s) 222, and/or a patient-input device, such as external device 24 or a patient device such as the patient's phone and/or computing device (e.g., to process stimulation-evoked signals sensed and/or accompanying signals captured from a patient different from patient 14, such as for aggregating certain responses for comparison). In the example of FIG. 2B, the processing of stimulation-evoked signals and/or accompanying signals occurs in a device other than IMD 200B. Referring again to FIG. 2A, the signal unit 216, discussed further below, receives information regarding stimulation-evoked signals and/or accompanying signals and may determine a quality of the stimulation-evoked signals. Signal unit 216 may, based on the determined quality of the stimulation-evoked signals, cause processing circuitry 210A to control the electrical stimulation circuitry 202 to deliver the electrical stimulation to the patient based on the received stimulation-evoked signals and/or accompanying signals. Indications of the received stimulation-evoked signals and/or accompanying signals may be stored in a storage device.

Processing circuitry 210A and/or 210B controls stimulation generation circuitry 202 to generate and apply the stimulation signals to selected combinations of electrodes 232A, 232B. In some examples, stimulation generation circuitry 202 includes a switch circuit (instead of, or in addition to, switch circuitry 204) that may couple stimulation signals to selected conductors within leads 230, which, in turn, deliver the stimulation signals across selected electrodes 232A, 232B. Such a switch circuit may selectively couple stimulation energy to selected electrodes 232A, 232B and to selectively sense bioelectrical neural signals of a sacral nerve or muscles of the patient with selected electrodes 232A, 232B. In other examples, however, stimulation generation circuitry 202 does not include a switch circuit and switch circuitry 204 does not interface between stimulation generation circuitry 202 and electrodes 232A, 232B. In these examples, stimulation generation circuitry 202 may include a plurality of pairs of current sources and current sinks, each connected to a respective electrode of electrodes 232A, 232B. In other words, in these examples, each of electrodes 232A, 232B is independently controlled via its own stimulation circuit (e.g., via a combination of a regulated current source and sink), as opposed to switching stimulation signals between different electrodes of electrodes 232A, 232B.

Storage device 212A and/or 212B may be configured to store information within IMD 200A and/or 200B, respectively, during operation. Storage device 212A and/or 212B may include a computer-readable storage medium or computer-readable storage device. In some examples, storage device 212A and/or 212B includes one or more of a short-term memory or a long-term memory. Storage device 212A and/or 212B may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, storage device 212A and/or 212B is used to store data indicative of instructions, e.g., for execution by processing circuitry 210A and/or 210B, respectively. As discussed above, storage device 212A and/or 212B is configured to store stimulation parameter settings 242.

Power source 224 is configured to deliver operating power to the components of IMD 200A and/or 200B. Power source 224 may include a battery and a power generation circuit to produce the operating power. In some examples, the battery is rechargeable to allow extended operation. In some examples, recharging is accomplished through proximal inductive interaction between an external charger and an inductive charging coil within IMD 200A and/or 200B. Power source 224 may include any one or more of a plurality of different battery types, such as nickel cadmium batteries and lithium ion batteries.

In some examples as shown in FIG. 2A, the processing circuitry 210A of the IMD 200A directs delivery of electrical stimulation by the electrodes 232A, 232B of leads 230A, 230B, receives stimulation-evoked signal data and/or accompanying signal data, e.g., from electrodes 232A, 232B and/or sensors 222, and generates output based on the received data and/or information. The signal unit 216 may use stimulation-evoked signals and/or accompanying signals, e.g., both of which may comprise stimulation-evoked signal data 254, and/or related information to determine a quality of stimulation-evoked signals and/or develop recommended electrical stimulation parameters or adjustments. Such recommendations may be output to a user, where the user can use the one or more recommended stimulation parameters to program the IMD 200A, e.g., by selecting or accepting the recommendations as stimulation parameter settings to be used by IMD 200A. For example, a particular cycling and/or a set of stimulation parameters are recommended to a user and presented to the user via the programmer. The user may accept the recommended cycling and/or one or more recommended stimulation parameters, and the programmer programs IMD 200A to implement and deliver stimulation with the selected electrode combination and/or stimulation parameters.

Processing circuitry 210A and/or 210B controls stimulation circuitry 202 to deliver stimulation energy with stimulation parameters specified by one or more stimulation parameter settings 242 stored on storage device 212A and/or 212B and, in the example of FIG. 2A, to collect stimulation-evoked signals and/or accompanying signals pertaining to the stored stimulation parameter settings 242. Processing circuitry 210A and/or 210B collects this stimulation-evoked signal and/or accompanying signals information by receiving the information via sensing circuitry 206 and/or directly from sensors 222. Processing circuitry 210A may also control stimulation generation circuitry 202 to test different parameter settings and record one or more corresponding stimulation-evoked signals and/or accompanying signals for each selected combination, and test different parameter settings as they compare to one or more sensed stimulation-evoked signals and/or accompanying signals. For example, processing circuitry 210A directs stimulation generation circuitry 202 to deliver stimulation via a particular cycling and the signal unit 216 collects the corresponding stimulation-evoked signal data 254 from telemetry circuitry 208. The stimulation-evoked signal data 254 for this test may be stored in the storage device 212A. Processing circuitry 210A may adjust the previously tested cycling of the stimulation delivered via the electrode combination to a different cycling and collect the corresponding stimulation-evoked signal data 254 from sensors 222 and sensing circuitry 206 in response to stimulation with the adjusted cycling. The stimulation-evoked signal data 254 received for the stimulation at the changed stimulation parameter, such as cycling, may be saved in the storage device 212A and may be output to a user. The processing circuitry 210A may continue to shift the cycling by either increasing or decreasing the cycling frequency and/or cycling duty cycle, and record the respective stimulation-evoked signal data 254 which is stored on the storage device 212A, and information based on the stimulation-evoked signal data 254 may be output to a user. While the example of cycling is provided, processing circuitry 210A may direct stimulation circuitry 202 to step through various incremental settings of other stimulation parameters, such as electrode combination or configuration, electrode polarity, amplitude, pulse width, pulse shape, pulse frequency or pulse rate, or cycling and record respective stimulation-evoked signal data 254 for each stepped value. In one or more examples, processing circuitry 210A may direct stimulation circuitry to turn on for a certain period of time, and/or to turn off for a period of time, or to turn on at a certain time of day and record the respective stimulation-evoked signal data 254. Stimulation circuitry 202 may shift more than one stimulation parameter for each test and collect sensed stimulation-evoked signal data 254 for each of the multiple shifted stimulation parameters.

In some examples, the signal unit 216 processes the stimulation-evoked signal information and/or accompanying signal information to determine a confidence interval of the stimulation-evoked signal information. For example, signal unit 216 may determine a variance or variances of the stimulation-evoked signal information and may determine a confidence interval corresponding to the stimulation-evoked signal information. If the stimulation-evoked signal information is relatively highly variable, confidence for the stimulation-evoked signal information may be low, e.g., indicating that a signal-to-noise ratio (SNR) of the stimulation-evoked signal information is low. In some examples, if the confidence and/or SNR of the stimulation-evoked signal information is low, signal unit 216 may process and/or average the stimulation-evoked signal information over a longer period of time to reduce the noise/variance. Correspondingly, IMD 200A and/or 200B, respectively, may then monitor the stimulation-evoked signal information for a longer period of time before, e.g., to determine stimulation parameters that may improve patient symptoms, outcomes, or the like. If signal unit 216 determines a confidence and/or SNR to be low, e.g., below a confidence and/or SNR threshold, IMD 200A and/or 200B may not change or base stimulation parameters on the stimulation-evoked signal information, e.g., default parameter settings and/or values may be used instead. Conversely, if the stimulation-evoked signal information has a relatively low variability, signal unit 216 may determine the confidence for the stimulation-evoked signal information to be relatively high, e.g., indicating a relatively high SNR of the stimulation-evoked signal information. IMD 200A and/or 200B may then operate on a pulse-by-pulse basis, e.g., delivering changes to stimulation parameters and/or sensing stimulation-evoked signal information more frequently, and signal unit 216 may extract features from the stimulation-evoked signal information over shorter periods of time and/or more frequently.

In some examples, the signal unit 216 processes the stimulation-evoked signal information and/or accompanying signal information to perform closed-loop control of the stimulation parameters based on the stimulation-evoked signal information and/or accompanying signal information. The signal unit 216 may process and/or store the stimulation-evoked signal information and/or accompanying signal information as stimulation-evoked signal data 254 in storage device 212A. For example, signal unit 216 may select or adjust one or more settings of parameter values, such as electrode combination or configuration, electrode polarity, amplitude, pulse width, pulse shape, pulse frequency or pulse rate, or cycling in response to stimulation-evoked signal data 254. The stimulation-evoked signal data 254 may be collected when electrical stimulation is not delivered, e.g., just after electrical stimulation is turned off, or upon delivery of electrical stimulation.

In some examples, the processing circuitry 210A and/or 210B of the IMD 200A and/or 210B, respectively, directs delivery of electrical stimulation of the electrodes 232A, 232B, and receives stimulation-evoked signal data 254 from one or more sensors 222, either directly (e.g., in the case of processing circuitry 210A) or via external controller (e.g., in the case of processing circuitry 210B), determines a quality of one or more stimulation-evoked signals, and controls the delivery of electrical stimulation of the electrodes 232A, 232B based on the stimulation-evoked signal data 254 and/or determined quality in a closed loop setting. The stimulation-evoked signal data 254 may be received via the telemetry circuitry 208 either directly or indirectly from sensors 15, 22 (FIG. 1). In an example, the IMD 200A and/or IMD 200B may receive the stimulation-evoked signal data 254 from an intermediate device other than sensors 15, 22, such as external device 24.

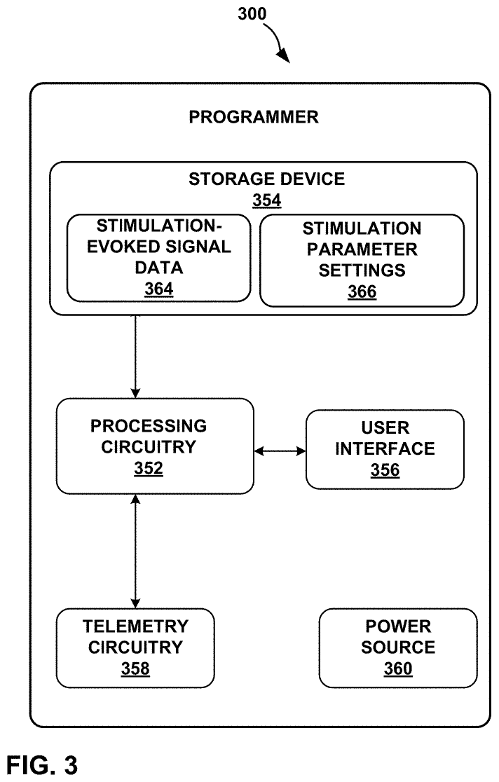

FIG. 3 is a block diagram illustrating an example configuration of components of an example external programmer 300. External programmer 300 may be an example of external device 24 of FIG. 1. Although external programmer 300 may generally be described as a hand-held device, such as a tablet computer or smartphone-like device, external programmer 300 may be a larger portable device, such as a laptop computer, or a more stationary device, such as a desktop computer. In addition, in other examples, external programmer 300 may be included as part of an external charging device or include the functionality of an external charging device, e.g., to recharge a battery or batteries associated with IMD 200. As illustrated in FIG. 3, external programmer 300 may include processing circuitry 352, storage device 354, user interface 356, telemetry circuitry 358, and power source 360. In some examples, storage device 354 may store instructions that, when executed by processing circuitry 352, cause processing circuitry 352 and external programmer 300 to provide the functionality ascribed to external programmer 300 throughout this disclosure. Each of these components, circuitry, or modules, may include electrical circuitry that is configured to perform some, or all of the functionality described herein. For example, processing circuitry 352 may include processing circuitry configured to perform the processes discussed with respect to processing circuitry 352.

In general, external programmer 300 includes any suitable arrangement of hardware, alone or in combination with software and/or firmware, to perform the techniques attributed to external programmer 300, and processing circuitry 352, user interface 356, and telemetry circuitry 358 of external programmer 300. In various examples, processing circuitry 352, telemetry circuitry 358, or other circuitry of external programmer 300 may include one or more processors, such as one or more microprocessors, DSPs, ASICs, FPGAs, or any other equivalent integrated or discrete logic circuitry, as well as any combinations of such components. External programmer 300 also, in various examples, may include a storage device 354, such as RAM, ROM, PROM, EPROM, EEPROM, flash memory, a hard disk, a CD-ROM, including executable instructions for causing the one or more processors to perform the actions attributed to them. Moreover, although processing circuitry 352 and telemetry circuitry 358 are described as separate modules, in some examples, processing circuitry 352 and telemetry circuitry 358 are functionally integrated. In some examples, processing circuitry 352, telemetry circuitry 358 or other circuitry of external programmer 300 may correspond to individual hardware units, such as ASICs, DSPs, FPGAs, or other hardware units.

The processing circuitry 352 is configured to direct delivery of electrical stimulation, receive information relating to one or more stimulation-evoked signals and/or accompanying signals. In some examples, the processing circuitry 352 is configured to control the electrical stimulation circuitry to deliver the electrical stimulation based on the received stimulation-evoked signals and/or a determined quality of the stimulation-evoked signals in a closed loop basis by directing the IMD to use particular stimulation parameters.

In some examples, storage device 354 may include instructions that cause processing circuitry 352 to obtain a parameter set from memory or receive user input and send a corresponding command to IMD 200, or instructions for any other functionality. In addition, storage device 354 may include a plurality of programs, where each program includes a parameter set that defines therapy stimulation or control stimulation. Storage device 354 may also store data received from a medical device (e.g., IMD 16) and/or a remote sensing device. For example, storage device 354 may store data recorded at a sensing module of the medical device, and storage device 354 may also store data from one or more sensors of the medical device, e.g., sensors 15, 22, 222, and/or electrodes 19, 21, 29, 232A, 232B. In an example, storage device 354 may store data recorded at a remote sensing device such as one or more stimulation-evoked signal and/or accompanying signal sensed by one or more electrodes and/or sensors.

User interface 356 may include a button or keypad, lights, a speaker for voice commands, a display, such as a liquid crystal (LCD), light-emitting diode (LED), or organic light-emitting diode (OLED). In some examples, the display includes a touch screen. User interface 356 may be configured to display any information related to the delivery of electrical stimulation including output, for example, information based on one or more stimulation-evoked signal. User interface 356 may also receive user input (e.g., indication of when the patient perceives stimulation, or a pain score perceived by the patient upon delivery of stimulation) via user interface 356. The user input may be, for example, in the form of pressing a button on a keypad or selecting an icon from a touch screen. The input may request starting or stopping electrical stimulation, the input may request a new electrode combination or a change to an existing electrode combination, or the input may request some other change to the delivery of electrical stimulation, such as a change in electrode combination or configuration, electrode polarity, amplitude, pulse width, pulse shape, pulse frequency or pulse rate, or cycling.

Telemetry circuitry 358 may support wireless communication between the medical device and external programmer 300 under the control of processing circuitry 352. Telemetry circuitry 358 may also be configured to communicate with another computing device via wireless communication techniques, or direct communication through a wired connection. In some examples, telemetry circuitry 358 provides wireless communication via an RF or proximal inductive medium. In some examples, telemetry circuitry 358 includes an antenna, which may take on a variety of forms, such as an internal or external antenna.

Examples of local wireless communication techniques that may be employed to facilitate communication between external programmer 300 and IMD 16 include RF communication according to the 802.11 or Bluetooth® specification sets or other standard or proprietary telemetry protocols. In this manner, other external devices may be capable of communicating with external programmer 300 without needing to establish a secure wireless connection. As described herein, telemetry circuitry 358 may be configured to transmit a spatial electrode movement pattern or other stimulation parameters to IMD 16 for delivery of electrical stimulation therapy.

Power source 360 is configured to deliver operating power to the components of external programmer 300. Power source 360 may include a battery and a power generation circuit to produce the operating power. In some examples, the battery is rechargeable to allow extended operation. Recharging may be accomplished by electrically coupling power source 360 to a cradle or plug that is connected to an alternating current (AC) outlet. In addition, recharging may be accomplished through proximal inductive interaction between an external charger and an inductive charging coil within external programmer 300. In other examples, traditional batteries (e.g., nickel cadmium or lithium ion batteries) may be used. In addition, external programmer 300 may be directly coupled to an alternating current outlet to operate.

In some examples, the external programmer 300 directs delivery of electrical stimulation of an IMD, receives information relating to stimulation-evoked signals and/or accompanying signals, and generates output based on the received information, e.g., a quality of sensed stimulation-evoked signals, an evaluation of efficacy of stimulation parameters, recommendations or other information to assist a user in programming stimulation parameters for delivery of electrical stimulation, information used as part of a closed loop control scheme to automatically adjust stimulation parameter, information to be recorded and/or tracked, and/or information or output which may be suitable for any other purpose relating to delivery of electrical stimulation therapy.

Programmer 300 may be a patient programmer or a clinician programmer and receives stimulation-evoked signal information and/or accompanying signal information, both of which may comprise stimulation-evoked signal data 364. Programmer 300 receives stimulation-evoked signal data 364 and allows a user to interact with the processing circuitry 352 via user interface 356 in order to identify efficacious parameter settings, such as cycling and/or one or more other stimulation parameters using the stimulation-evoked signal data 364. Programmer 300 further assists the user in programming a neurostimulation device by using the stimulation-evoked signal data 364 displayed on the user interface 356. In addition, programmer 300 may be used as part of a closed loop control scheme to automatically adjust stimulation parameters based at least on stimulation-evoked signal data 364. In some examples, programmer 300 receives stimulation-evoked signal data 364 from one or more sensor devices, e.g., sensors 15, 22, 222, and stores the stimulation-evoked signal data 364 in the storage device 354. In some examples, programmer 300 may be device specifically made to communicate with an IMD, e.g., IMD 16, IMD 200A, IMD 200B, and the like, as part of an electrical stimulation system. In other examples, programmer 300 may be a device configured to interact with an IMD or other device of an electrical stimulation system, e.g., a computing device and/or mobile phone configured to run suitable application software for the electrical stimulation system and configured to communicate with one or more devices, e.g., an IMD, of the electrical stimulation system.

Programmer 300 may be used to determine a quality of sensed stimulation-evoked signals resulting from particular parameter settings of the IMD by testing parameter settings and recording stimulation-evoked signal data 364 for each parameter setting. For example, processing circuitry 352 may cause the IMD to scan through any of the parameter settings, e.g., amplitude, frequency, or the like, sequentially, e.g., from 0 milliamps (mA) to 5 mA in 0.1 mA increments, or randomly, or via sweeping by increasing and/or decreasing the parameter setting value, e.g., oscillating relatively quickly between increasing and decreasing in order to determine one or more threshold values of one or more parameter settings. In some examples, programmer 300 may be used to cause the IMD to automatically scan though a plurality of electrode combinations or parameter combinations. Processing circuitry 352 causes the IMD to automatically scan through each of a plurality of parameter combinations, including electrode combinations and parameter combinations. For each combination, the programmer 300 obtains and records stimulation-evoked signal data 364. In some examples, programmer 300 may be used to cause the IMD to automatically scan through a plurality of electrode combinations or parameter combinations at one or more times, e.g., periodically every hour, day, week, month, year, and/or non-periodically, e.g., according to a schedule or other determination of when to repeat a scan, and obtains and records stimulation-evoked signal data 364 for each scan. In some examples, programmer 300 or another device, e.g., IMD 16, external device 24, server 26, or other device, may compare the recorded stimulation-evoked signal data 364 over time.

Alternative to or in addition to the automatic scanning process, the user could manually advance scanning through electrode pairs and/or parameter combinations, for example with an arrow button on user interface 356. For example, a user may scan through the electrode pairs or parameter combinations to test and record stimulation-evoked signal data 364.

Processing circuitry 352 controls stimulation circuitry 202 to deliver stimulation energy with stimulation parameters specified by one or more stimulation parameter settings 366 stored on storage device 354, and to collect stimulation-evoked signal information pertaining to the stored stimulation parameter settings 366. Processing circuitry 352 may also control stimulation circuitry 202 to test different parameter settings and record one or more corresponding stimulation-evoked signal for each selected combination, and test different parameter settings as they compare to one or more stimulation-evoked signal. For example, processing circuitry 352 directs stimulation circuitry 202 to deliver stimulation with a particular cycling and one or more stimulation-evoked signal is collected from telemetry circuitry 358. The stimulation-evoked signal data 364 for this test may be stored in the storage device 354.

Processing circuitry 352 may be configured to shift the previously tested cycling to a different cycling and collect stimulation-evoked signal data 364, which may be saved in the storage device 354. The processing circuitry 352 may continue to shift the cycling by either increasing or decreasing the cycling (e.g., the cycling frequency and/or cycling duty cycle), and record the respective stimulation-evoked signal data 364. Information relating to the stimulation-evoked signal data 364 may then be output, e.g., to a different device for processing and/or may be output via user interface 356. While the example of cycling is provided, processing circuitry 352 may direct stimulation circuitry to step through various incremental settings of other stimulation parameters, such as stimulation amplitude, stimulation pulse width, or stimulation frequency, and record the respective stimulation-evoked signal data 364 for each stepped value. Stimulation circuitry 202 may shift more than one stimulation parameter for each test and collect stimulation-evoked signal data 364 for the multiple shifted stimulation parameters.

In some examples, the processing circuitry 352 of programmer 300 directs delivery of electrical stimulation of the electrodes 232A, 232B, receives stimulation-evoked signal data 364, determines a quality of stimulation-evoked signals, and controls the delivery of electrical stimulation of the electrodes 232A, 232B based on the received stimulation-evoked signal data 364 and/or determined quality in a closed loop setting. The stimulation-evoked signal data 364 may be received via the telemetry circuitry 358 either directly or indirectly from sensor(s) 222 and/or a patient-input device.

The architecture of external programmer 300 illustrated in FIG. 3 is shown as an example. The techniques as set forth in this disclosure may be implemented in the example external programmer 300 of FIG. 3, as well as other types of systems not described specifically herein. Nothing in this disclosure should be construed so as to limit the techniques of this disclosure to the example architecture illustrated by FIG. 3.

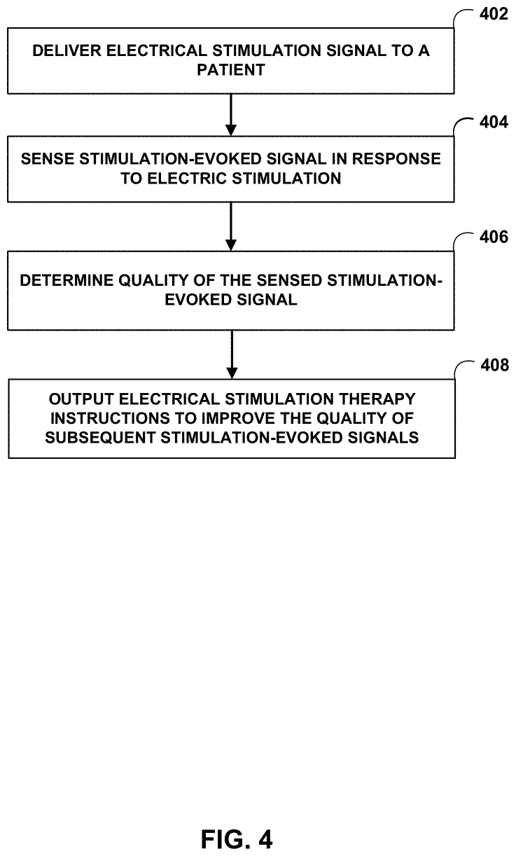

FIG. 4 is a flow diagram illustrating an example method of operation, in accordance with one or more techniques of this disclosure. Although FIG. 4 is discussed with reference to IMD 200A of FIG. 2A and external programmer 300 of FIG. 3, it is to be understood that the methods discussed herein may include and/or utilize other systems and methods in other examples.

IMD 200A may deliver one or more electrical stimulation signals to a patient (402). For example, processing circuitry 210A may control stimulation circuitry 202 to deliver stimulation energy via electrodes 232A, 232B with stimulation parameters specified by one or more stimulation parameter settings 242 stored on storage device. In some examples, the electrical stimulation signal may be delivered to one or more of a sacral nerve, a saphenous nerve, a sciatic nerve, a tibial nerve, or a pudendal nerve in any combination. In other examples, the electrical stimulation signal may be delivered to any other nerve or muscle, any portion of the patient's brain, any organ of the patient, or any other tissue of the patient.

In some examples, delivering the one or more electrical stimulation signal to the patient comprises delivering one or more stimulation signal having one or more of non-equal pulse amplitudes, non-equal pulse durations, non-equal polarities, or non-equal pulse frequencies.

In some examples, IMD 200A may deliver one or more electrical stimulation signals to the patient for modulating bladder and/or bowel control comprises delivering the one or more electrical stimulation signals to the patient for a pelvic health disorder, bladder dysfunction, retention, overactive bladder, urgency, urgency frequency, urinary incontinence, bladder incontinence, fecal incontinence, stress urinary incontinence, urinary retention, sexual dysfunction, obesity, gastroparesis, intractable constipation, pelvic pain, chronic pain, bladder pain syndrome, irritable bowel syndrome, inflammatory bowel disease, interstitial cystitis, neurogenic bowel, neurogenic bladder, neurological disorders, tremor, Parkinson's disease, epilepsy, multiple sclerosis, stroke, spinal cord injury, neuropathy, or the like.. In some examples, IMD 200A may deliver one or more electrical stimulation signals to a sacral nerve, a tibial nerve, a saphenous nerve, a pudendal nerve, and/or a sciatic nerve, and/or other nerves and all branches of such nerves of the patient. In some examples, IMD 200A may deliver one or more electrical stimulation signals to the patient in a non-clinic location and/or environment, e.g., at home.

Electrodes 232 and/or sensors 222 may sense one or more stimulation-evoked signals (404). The stimulation-evoked signals sensed by electrodes 232 and/or sensors 222 may be generated by one or more signal sources in response to the delivered electrical stimulation signals. In some examples, the stimulation-evoked signal may comprise a composite stimulation-evoked signal comprising a composite of signals generated by one or more signal sources in response to the delivered electrical stimulation signals. For example, a stimulation-evoked signal sensed by electrodes 232 and/or sensor(s) 222 may be a composite of a plurality of stimulation-evoked signals, each of which may originate from the same or a different signal source (e.g., muscle, nerve, etc.), each of which may originate at the same time or at a different time, and each of which may have the same or different duration. In some examples, processing circuitry 210A may control stimulation circuitry 202, telemetry circuitry 208, sensing circuitry 206, electrodes 232, and/or sensors 222 to collect stimulation-evoked signal information, e.g., stimulation-evoked signal data 254. Processing circuitry 210A may store received stimulation-evoked signal data 254 in storage device 212A. In some examples, IMD 200A may receive stimulation-evoked signal(s) as one or more of physiological signals. In some examples, a stimulation-evoked signal may include one or more of an EMG, an ECAP, MMG, a network excitability, and the like.

In some examples, a signal source may comprise a muscle, a nerve, and/or any combination thereof, e.g., in the case of composite stimulation-evoked signals. In some examples, e.g., including composite stimulation-evoked signals, at least one signal source may be located relatively near to the sensor capturing the stimulation-evoked signal(s), e.g., an ECAP signal generated by an ECAP signal source located within about 10 mm from the sensor. In other examples including composite stimulation-evoked signals, at least one of two or more signal sources may be located relatively far from the sensor capturing the stimulation-evoked signal(s). For example, at least one of the two or more signal source may be least 5 millimeters (mm) from electrodes 232 and/or a sensor 222, at least 10 mm from electrodes 232 and/or a sensor 222, at least 100 mm from electrodes 232 and/or a sensor 222, at least 200 mm from electrodes 232 and/or a sensor 222, at least 1 meter from electrodes 232 and/or a sensor 222, or any other distance within patient 14 from electrodes 232 and/or a sensor 222. As a result, stimulation-evoked signals from the two or more signal sources and captured as composite stimulation-evoked signals may arrive at the electrodes 232 and/or a sensor 222 at different times, e.g., there may be a signal capture time delay between the signals from each source being captured by electrodes 232 and/or a sensor 222. Additionally, different signal sources may have different response times, e.g., differing time delays between electrical stimulation beginning or ending and the initiation of a response. As a result, there may be a signal capture time delay because of the differing response time delays, and/or the signal capture delay may be a combination of the different distances and different response times of the two or more signal sources.

In some examples, the composite stimulation-evoked signal that includes two or more stimulation-evoked signals from the one or more signal sources may have a relative long duration, e.g., at least 1 ms, at least 3 ms, at least 5 ms, at least 10 ms, at least 20 ms, etc. For example, the composite stimulation-evoked signal may comprise an ECAP signal generated relatively quickly after delivery of electrical stimulation signals, e.g., within about 0.5 ms, or within about 1 ms, or within about 3 ms, or within about 5 ms, or within about 10 ms, and an EMG signal generated relatively slowly after delivery of electrical stimulation signals, e.g., after 5 ms, or after 3 ms, or after 1 ms. In some examples, the composite stimulation-evoked signal may comprise signals from multiple signal sources that do not overlap in time. For example, the composite stimulation-evoked signal may comprise an ECAP signal from a signal source relatively close to the sensor and/or electrode followed by an EMG signal or another ECAP signal from the same signal source (or reflex ECAP), or from a different signal source that may be relatively far from the sensor and/or electrode, e.g., such that the ECAP from the close signal source is no longer present while the EMG signal and/or ECAP from the more distant signal source are received by the sensor and/or electrode. In some examples, the composite stimulation-evoked signal may have an amplitude of one or more peaks that are greater than 1 millivolt (mV), or greater than 0.1 mV, greater than 0.01 mV, or greater than 0.001 mV.

In some examples, processing circuitry 210A may receive one or more sensed stimulation-evoked signals, e.g., a stimulation-evoked signal from a signal source and/or a composite stimulation-evoked signal. For example, processing circuitry 210A may receive one or more composite stimulation-evoked signals from sensing circuitry 206, and may store the one or more composite stimulation-evoked signal and any other information relating to the one or more composite stimulation-evoked signal in a storage device, e.g., as stimulation-evoked signal data 254. For example, processing circuitry 210A may store a stimulation-evoked signal as digital information representing a signal amplitude at a plurality of times. In some examples, the signal amplitude may represent a sensed voltage, current, capacitance, or inductance, e.g., for an electrical signal sensor. In some examples, the signal amplitude may represent a displacement, a pressure, accelerometer data, a sound, e.g., such as an MMG signal. In still other examples, the signal amplitude may represent any measurable physical quantity representing a physiological response of a signal source (e.g., muscle, nerve, and the like) to electric stimulation.

In some examples, there may be a signal capture time delay because of the differing response time delays, and/or the signal capture delay may be a combination of the different distances and different response times of the two or more signal sources. In some examples, the composite stimulation-evoked signal that includes stimulation-evoked signals from the two or more signal sources may have a relative long duration, e.g., at least 5 ms, at least 10 ms, at least 20 ms, etc. For example, the composite stimulation-evoked signal may comprise an ECAP signal generated relatively quickly after delivery of electrical stimulation signals, e.g., within 10 ms, and an EMG signal generated relatively slowly after delivery of electrical stimulation signals, e.g., after 5 ms, or after 3 ms, or after 1 ms. In some examples, the composite stimulation-evoked signal may comprise signals from multiple signal sources that do not overlap in time. For example, the composite stimulation-evoked signal may comprise an ECAP signal from a signal source relatively close to the sensor and/or electrode followed by an EMG signal or another ECAP signal from the same signal source, or from a different signal source that may be relatively far from the sensor and/or electrode, e.g., such that the ECAP from the close signal source is no longer present while the EMG signal and/or ECAP from the more distant signal source are received by the sensor and/or electrode. In some examples, the composite stimulation-evoked signal may have an amplitude of one or more peaks that are greater than 1 millivolt (mV), or greater than 0.1 mV, greater than 0.01 mV or greater than 0.001 mV.

In some examples, processing circuitry 210A may receive stimulation-evoked signals, e.g., from sensing circuitry 206, and may store the stimulation-evoked signals and any other information relating to the stimulation-evoked signals in a storage device, e.g., as stimulation-evoked signal data 254. In some examples, processing circuitry 210A may receive accompanying signals, e.g., from sensors 222, and may process and/or store the accompanying signals and any other information relating to the accompanying signals in the storage device, e.g., also as stimulation-evoked signal data 254. In some examples, processing circuitry 210A may store a stimulation-evoked signal and/or accompanying signal as digital information representing a signal amplitude at a plurality of times. In some examples, the signal amplitude may represent a sensed voltage, current, capacitance, or inductance, e.g., for an electrical signal sensor. In some examples, the signal amplitude may represent a displacement, a pressure, accelerometer data, a sound, e.g., such as an MMG signal. In still other examples, the signal amplitude may represent any measurable physical quantity representing a physiological response of a signal source (e.g., muscle, nerve, and the like) to electrical stimulation.

IMD 200A, external device 24, and/or server 26 may determine a quality of the one or more sensed stimulation-evoked signals (406). For example, IMD 200A may determine the quality of stimulation-evoked signals based on determining features of the stimulation-evoked signals and/or based on additional information such as one or more accompanying signals from one or more other sensors 222 that are sensed at or near the time of delivery of electrical stimulation signals and/or sensing of the stimulation-evoked signals. For example, the quality of a stimulation-evoked signal may be determined based on a stimulation-evoked signal alone, based on an accompanying signal alone, or based on both.

In some examples, IMD 200A may determine a quality of sensed stimulation-evoked signals based the stimulation-evoked signals. For example, IMD 200A may process a stimulation-evoked signal and determine one or more features of the stimulation-evoked signal. The one or more features may include a signal strength, the presence, amount, and/or characteristics of noise, one or more signal artifacts, an amplitude of the stimulation-evoked signal, an area of at least a portion of the stimulation-evoked signal (e.g., an amount of the signal integrated over a period of time), one or more frequency components and/or the spectral content of the stimulation-evoked signal, the identification of one or more peaks and/or peak characteristics such as peak height/amplitude, area, number of peaks, time between one or more peaks, peak width, corresponding negative peaks and peak characteristics (e.g., valleys), a signal growth and/or growth curve, a latency of a peak and/or valley (e.g., time-delay between delivery of an electrical stimulation signal and the time at which the peak/valley is sensed), an impedance and/or impedance change of one or more leads 230 and/or one or more electrodes 232, a standard deviation of at least a portion of the stimulation-evoked signal or a standard deviation of a plurality of stimulation-evoked signals, a power spectrum and/or power spectral density of the stimulation-evoked signal, or any other suitable signal feature.

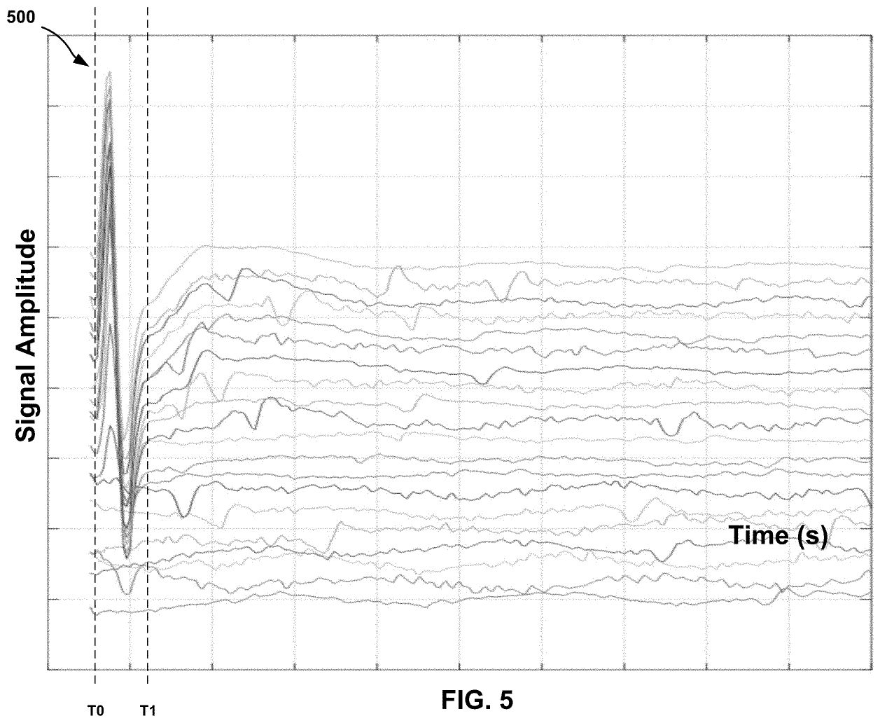

As an example, IMD 200A may determine a quality of a stimulation-evoked signal based on SNR of the stimulation-evoked signal. For example, the SNR of the stimulation-evoked signal may be based on a mean and a standard deviation of at least a portion of the stimulation-evoked signal or of a plurality of stimulation-evoked signals, e.g., as further described below with reference to FIGS. 6-9. In another example, IMD 200A may determine one or more signal artifacts of the stimulation-evoked signal and may determine a quality of a stimulation-evoked signal based on the determined artifacts. For example, IMD 200A may identify and/or determine a spurious signal corresponding to a spurious EMG signal from a muscle, e.g., either due to patient movement, a patient position, and/or a patient physiological state (e.g., full bladder, voiding event, etc.), such as illustrated in FIGS. 5 and/or 10 below. IMD 200A may then determine a quality of the stimulation-evoked signal based on the identified spurious artifact. For example, the spurious artifact(s) may or may not interfere with identification and/or determination of the response of one or more signal sources based on the stimulation-evoked signal that includes the spurious artifact(s), and IMD 200A may determine the quality accordingly. In some examples, IMD 200A may determine one or more artifacts based on a standard deviation being greater than a threshold standard deviation or a SNR being greater than a threshold SNR.

In some examples, IMD 200A may determine a quality of sensed stimulation-evoked signals based on a sensed accompanying signal. For example, IMD 200A may process an accompanying signal and determine one or more features of the accompanying signal. The one or more features may include a signal strength, the presence, amount, and/or characteristics of noise, one or more signal artifacts, an amplitude of the accompanying signal, an area of at least a portion of the accompanying signal (e.g., an amount of the signal integrated over a period of time), one or more frequency components and/or the spectral content of the accompanying signal, the identification of one or more peaks and/or peak characteristics such as peak height/amplitude, area, number of peaks, time between one or more peaks, peak width, corresponding negative peaks and peak characteristics (e.g., valleys), a signal growth and/or growth curve, a latency of a peak and/or valley (e.g., time-delay between delivery of an electrical stimulation signal and the time at which the peak/valley is sensed), a standard deviation of at least a portion of the accompanying signal or of a plurality of accompanying signals, a power spectrum and/or power spectral density of the accompanying signal, or any other suitable signal feature.

As an example, IMD 200A may determine a quality of a stimulation-evoked signal based on SNR of an accompanying signal. For example, the SNR of the accompanying signal may be based on a mean and a standard deviation of at least a portion of the accompanying signal or of a plurality of accompanying signals. In another example, IMD 200A may determine one or more signal artifacts of the accompanying signal and may determine a quality of a stimulation-evoked signal based on the determined artifacts. For example, IMD 200A may identify and/or determine a spurious signal/event within the accompanying signal, e.g., peaks or other features which may indicate a patient posture (e.g., movement from accelerometer data or a physiological state such as voiding from pressure sensor data). IMD 200A may then determine a quality of the stimulation-evoked signal based on the identified spurious signal of an accompanying signal. For example, the spurious signal(s) may or may not be associated with a known quality (e.g., good/bad) of stimulation-evoked signals, e.g., a spurious signal of accelerometer data indicate patient motion which may be known to be associated with poor stimulation-evoked signal data.

In some examples, one or more signal artifact, e.g., of a stimulation-evoked signal and/or an accompanying signal, may be a motion artifact, electrical interference (e.g., from a signal source within the patient and/or from the environment in which the patient is in during therapy and/or sensing of stimulation-evoked signals), a cardiac artifact (e.g., a change in heart rate), a bowel fullness, a bladder fullness, a spurious EMG, and the like.

In some examples, IMD 200A may determine a quality of a stimulation-evoked signal based determining that a patient is in a predetermined posture at the time the electrical stimulation signals are delivered and/or at the time the one or more stimulation-evoked signals are sensed. For example, IMD 200A may determine that the patient is in a particular position, determine a movement and/or motion of the patient, and/or determine a physiological state of the patient (e.g., increased heart rate, full bladder, etc.). In some examples, the predetermined posture may include one or more of sitting down, standing, or lying down.

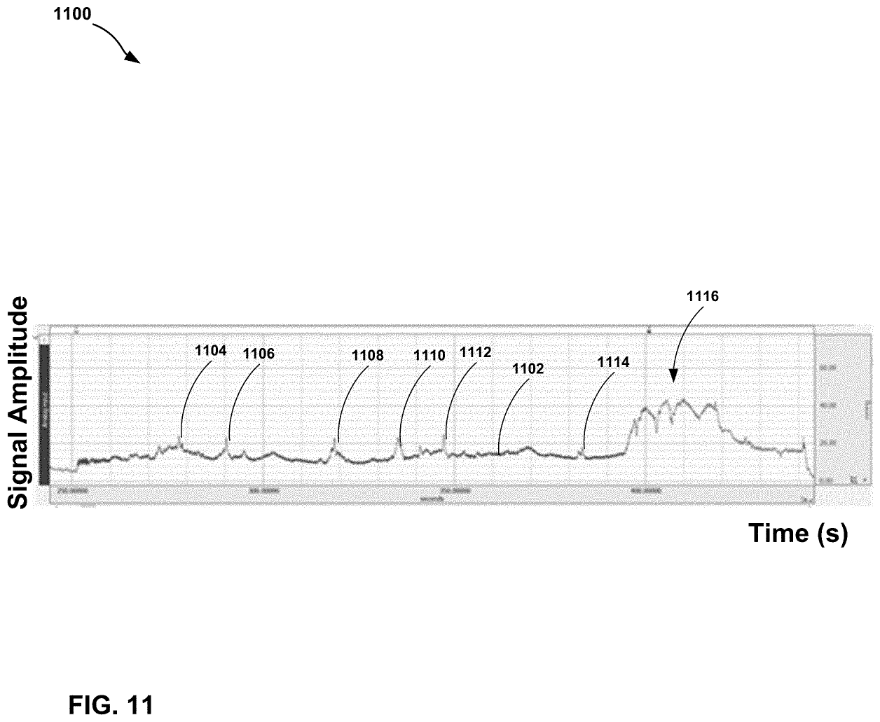

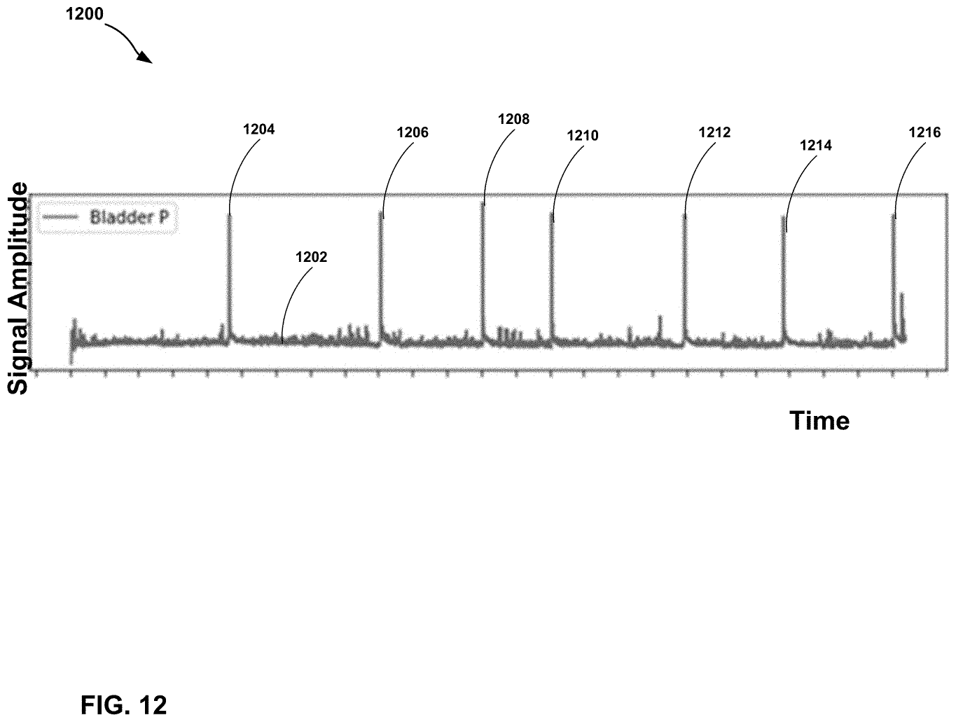

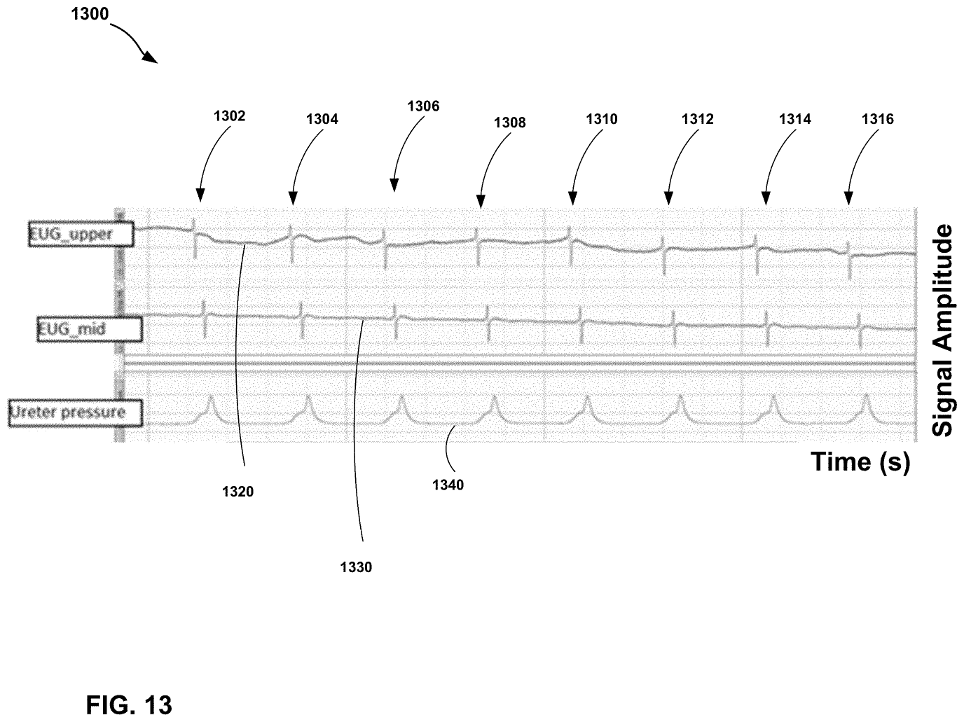

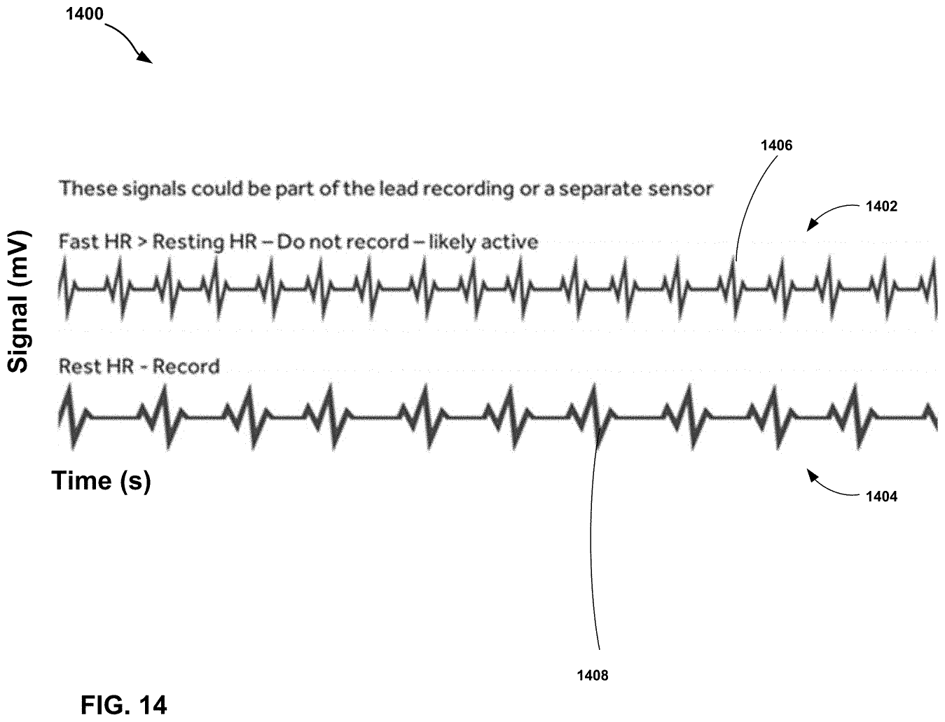

Examples of accompanying signals may include a signal from an accelerometer configured to indicate patient movement and/or posture, a signal from a heart rate monitor (an example of which is illustrated in FIG. 14 below) configured to indicate the heart rate of the patient (e.g., from which patient movement may be determined), a signal from a pressure sensor (an example of which is illustrated in FIG. 11 below), an impedance sensor (an example of which is illustrated in FIG. 12 below), an electro-ureterogram (EUG) sensor (an example of which is illustrated in FIG. 13 below), each of which may be configured to indicate the fullness of the patient's bladder and/or voiding events (e.g., from which a responsivity of certain signal sources, such as a detrusor muscle, may be inferred), or any other signal from any other sensor configured to determine a patient posture at or near the time of the delivery of electrical stimulation signals and/or sensing of the stimulation-evoked signals. In some examples, an accompanying signal may include an ultrasound signal, e.g., from an ultrasound sensor configured to determine fullness of a patient's bowel and/or bladder. In some examples, an accompanying signal may include a slow-wave EMG signal, e.g., from an EMG sensor configured to measure peristalsis. In some examples, an accompanying signal may include a signal from a toilet sensor, e.g., a toilet sensor configured to determine proximity of IMD 200A from which a voiding event, a bowel movement, or a patient position (e.g., sitting) may be determined and/or inferred. In some examples, an accompanying signal may include a “patient information signal,” e.g., information from which the quality of a stimulation-evoked signal may be determined and/or inferred such as a voiding diary, which may be in the form of an electronic voiding diary application.

In some examples, IMD 200A may determine the quality of the stimulation-evoked signal relative to a qualitative (e.g., poor, medium, good) and/or quantitative scale (e.g., 4 out of 10), which may be based on and/or relative to a known quality standard and/or benchmark. In some examples, IMD 200A may determine an uncertainty and/or uncertainty value/level of the stimulation-evoked signal. In some examples, IMD 200A may determine whether the stimulation-evoked signal may be used as the basis for further determinations and/or whether subsequent stimulation-evoked signals should be acquired to be used as the basis for further determinations. In some examples, IMD 200A may determine an uncertainty level, a quality level, and/or a reliability and/or trustworthiness of one or more determinations (e.g., that are made based on the stimulation-evoked signal) based on the determined quality of the stimulation-evoked signal.

IMD 200A, external device 24, and/or server 26 may output instructions to improve the quality of one or more subsequent stimulation-evoked signals (408). For example, IMD 200A may output instructions (e.g., for causing improvement in the quality of one or more subsequent stimulation-evoked signals) directing the patient to be in a particular posture, e.g., to stop moving, to sit, stand, lie down, and the like, and/or directing electrical stimulation to occur after a patient action and/or event, such as a voiding event. For example, IMD 200A may determine that the patient is in a first predetermined posture at (406), and may provide instructions directing the patient to be in a second predetermined posture that is different from the first predetermined posture during subsequent delivery of electrical stimulation, or directing the patient to change an electrical stimulation parameter, or directing the electrical stimulation device to change an electrical stimulation parameter.

IMD 200A may output instructions to direct the patient, clinician, and/or user of IMD 200A (or external device 24) to adjust and/or change the placement and/or positioning of leads 230A, 230B change one or more electrical stimulation therapy parameters, e.g., a particular combination of electrodes, polarities of selected electrodes, a voltage or current amplitude, a pulse width, a pulse frequency, and the like. In some examples, IMD 200A may output instructions to automatically change one or more electrical stimulation therapy parameters, e.g., to cause a patient programmer device to change one or more of the electrical stimulation therapy parameters.

In some examples, IMD 200A may output instructions to repeat a measurement, e.g., (402)-(406) automatically without notifying a user, with or without changing one or more electrical stimulation therapy parameters, e.g., after a period of time to wait for patient 14 to change posture. In some examples, IMD 200A may output instructions to automatically change one or more electrical stimulation therapy parameters, e.g., to cause a patient programmer device to change one or more of the electrical stimulation therapy parameters.

IMD 200A may output an alert to a patient at home that it is time for an annual checkup. IMD 200A may output instructions to the patient to sit (or be in a particular posture), e.g., via the patient's phone executing application software, which may guide the patient through stimulation and or measurements, e.g., (402)-(408). IMD 200A may then cause the method steps (402)-(408) to be executed and determine whether sensed stimulation-evoked signals are of sufficient quality (e.g., at (406)). If the quality is not sufficient, IMD 200A may instruct the patient to re-attempt the measurement. Once a signal of sufficient quality is obtained, IMD 200A may utilize one or more stimulation-evoked signals to adjust the therapy, maintain therapy, or instruct patient to consider scheduling an appointment.

FIG. 5 is a plot 500 of example stimulation-evoked signals including an artifact, in accordance with one or more techniques of this disclosure. In the example shown, plot 500 includes a plurality of EMG signals including a spurious signal (e.g., artifact) at time T0 and/or between times T0 and T1. For example, the spurious (e.g., non-stimulation-evoked) signal at time T0 (or between T0-T1) may be a sudden muscle contraction, e.g., a detrusor muscle.

FIGS. 6-9 are plots of example stimulation-evoked signals of varying quality. In the specific examples of FIGS. 6-9 below, each signal plotted represents a voltage amplitude of a circuit including an electrode 232 that varies in time in proportion to a time-varying electric field sensed by the electrode 232. The time-varying electric field in the examples shown is caused by one or more signal sources, e.g., nerve, muscle, or other tissue, of a patient in response to electrical stimulation. However, FIGS. 6-9 may generally represent one or more other quantities. In some examples, each signal plot may represent an amplitude as a function of time of a sensed quantity over time, the quantity varying in proportion to a physiological response of a signal source. In some examples, the quantity is an amplitude measured by a sensor. For example, the amplitude may be a voltage and/or current that varies in time according to an amplitude of an electric field and/or potential emitted and/or induced by a signal source. In some examples, stimulation-evoked signals 602-902 described below may each be a stimulation-evoked signal generated and/or sensed for a period of time between T0 and T1 as shown (e.g., a sensing “time window”).

FIG. 6 is a plot 600 of an example stimulation-evoked signal 602, in accordance with one or more techniques of this disclosure. In the example shown, signal 602 is a voltage amplitude that varies in time in proportion to a time-varying electric field sensed by an electrode 232, the time-varying electric field caused by a signal source in response to electrical stimulation. In the example shown, time T0 corresponds to a time at which electrical stimulation of a nerve or muscle ceases, e.g., is turned off, and time T1 corresponds to the ending time of the sensing time window, e.g., the sensing time window is the difference between T1 and T2. In some examples, signal 602 may have a signal length in time that is equal to the time window, e.g., the physiological response of the signal source emits a detectable quantity (e.g., electric field) that lasts for the length of time of the time window. In other examples, the signal length of signal 602 may be less than the time window. Generally, the time window may be chosen based on signal length, e.g., time T0 may be chosen to be the time at which electrical stimulation ceases and time T1 may be chosen based on the time-length of the sensed signal 602.

In the example shown, signal 602 includes signal features, e.g., a peak at time 606 and a valley at time 608 (which may be considered a “peak” with a negative amplitude and may be simply referred to as a “peak” herein) and a standard deviation 604. In the example shown, the standard deviation 604 as illustrated may be an “instantaneous” standard deviation at the particular time indicated in FIG. 6, but may still visually represent a standard deviation of signal 602 that may be determined over a portion or the entire time window, or determined based on a plurality of signals 602. In some examples, the SNR of signal 602 may be determined based, at least in part, on the determined standard deviation.

The example shown in FIG. 6 represents a signal 602 with both a “good,” e.g., relatively high signal strength and quality. For example, signal features such as peaks 604 and/or 608, and associated calculations such as peak width and integrated areas of the peaks, may be easily distinguishable, e.g., from noise and/or artifacts. For example, the standard deviation 604, e.g., representing noise, may be small enough in amplitude to result in a high, or good, SNR.

FIG. 7 is a plot 700 of another example stimulation-evoked signal 702, in accordance with one or more techniques of this disclosure. In the example shown, signal 702 is a voltage amplitude that varies in time in proportion to a time-varying electric field sensed by an electrode 232, the time-varying electric field caused by a signal source in response to electrical stimulation. In the example shown, signal 702 includes peaks at times 706 and 708 which may correspond in time to peaks 606 and 608 of FIG. 6. The example shown in FIG. 7 represents a signal 702 with a “good,” e.g., relatively high signal strength and a “poor,” e.g., relatively low quality. For example, signal features such as peaks 704 and/or 708 may be distinguishable, but associated calculations such as peak width and integrated areas of the peaks may be confounded by noise/and or irregularities and/or artifacts in signal 702. For example, the standard deviation 704, e.g., representing noise, may be larger than standard deviation 604 and signal 702 may have a lower SNR than signal 602.

FIG. 8 is a plot 800 of another example stimulation-evoked signal 802, in accordance with one or more techniques of this disclosure. The example shown in FIG. 8 represents a signal 802 with a “poor,” e.g., relatively low or zero signal strength. The noise of signal 802 may be relatively low, e.g., as illustrated by a relatively small standard deviation 804, however, the SNR and/or quality of signal 802 may be either high or low, e.g., there are no signal features so it may be difficult to tell whether there is a signal that has been captured. However, signal 802 may still be a high quality signal because information may be derived and/or determined based on signal 802. For example, with the low noise, if a signal feature in signal 802 is expected and there is none, that may indicate poor leads 230 placement, e.g., signal 802 is a high quality signal from which to make a determination with a relatively high degree of certainty (e.g., or a relative low uncertainty), and as such, signal 802 may be a “high” quality signal.

FIG. 9 is a plot 900 of another example stimulation-evoked signal 902, in accordance with one or more techniques of this disclosure. The example shown in FIG. 9 represents a signal 802 with both a “poor,” e.g., relatively low or zero signal strength and a “poor,” e.g., relative low quality. The noise of signal 902 may be relatively high, e.g., as illustrated by a relatively large standard deviation 904. By way of contrast with signal 802, information may be difficult and/or impossible derived and/or determined based on signal 902. For example, with the high noise, if a signal feature in signal 902 is expected and there is none, it may be difficult and or impossible to identify any signal features with a reasonable degree of certainty in order to make any determinations based on signal 902 and as such, signal 902 may be a “low” quality signal.

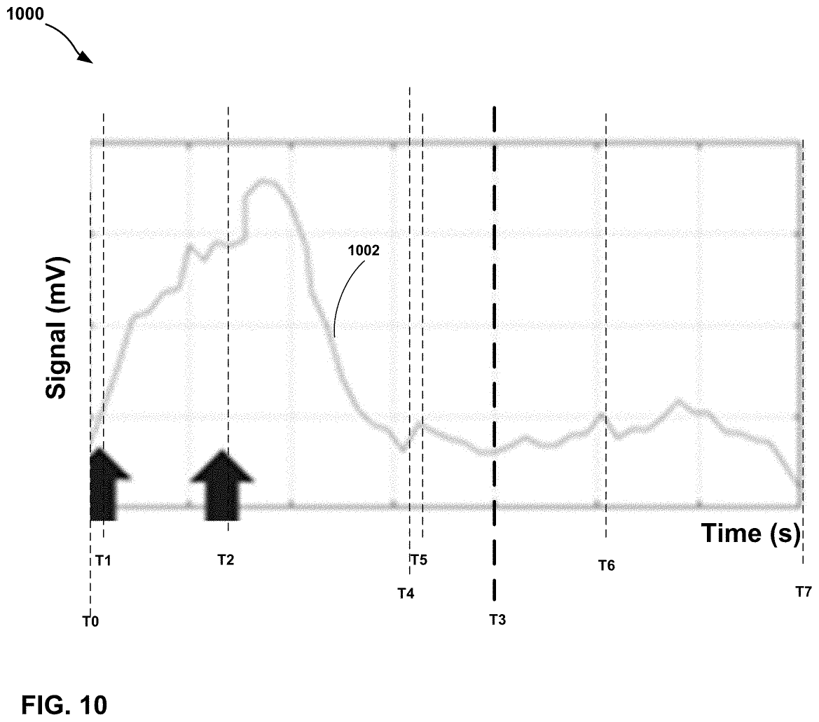

FIG. 10 is a plot 1000 of an example stimulation-evoked signal 1002 including an artifact, in accordance with one or more techniques of this disclosure. In the example shown, signal 1002 is a voltage amplitude that varies in time in proportion to a time-varying electric field sensed by an electrode 232 and/or sensor 222, the time-varying electric field caused by a signal source in response to electrical stimulation. In the example shown, the electrical stimulation may be a constant amplitude.

The example shown in FIG. 10 represents a stimulation-evoked signal 1002 including one or more artifacts. In the example shown, times T0 and T7 represent the time window of stimulation-evoked signal 1002 and the amount of time over which signal 1002 was captured and recorded. Signal features of signal 1002 before time T3, e.g., the increased amplitude and any other features of signal 1002 between times T0 and T3 may be response to patient movement, e.g., at times T1 and T2. In the example shown, signal 1002 may not contain any signal features from which to make a determination that the patient moved at or near times T1 and T2, and it may not be possible to determine whether signal features between times T0 and T2 are due to patient movement or responses of signal sources. However, IMD 200A may determine that patient movement occurred at times T1 and T2 based on an accompanying signal, e.g., received from a sensor 222 such as an accelerometer (not shown). In the example shown, IMD 200A may determine the quality of signal 1002 based on the accompanying signal, e.g., IMD 200A may determine that signal 1002 is a low quality signal between times T0 and T3 because of the determined patient movement at or near those times, namely, at times T1 and T2.

In some examples, IMD 200A may determine the quality of signal 1002 based on an accompanying signal (e.g., the accelerometer signal) alone, e.g., because it may be known that patient movement affects signal 1002 as shown. In other examples (not shown), patient movement may prevent determination of responses of signal sources to delivered electrical stimulation because the response of signal 1002 due to patient movement overwhelms any stimulation-evoked signal from a signal source response. In other words, IMD 200A may capture signal 1002 and determine it to be low quality based on an accompanying signal without processing and/or evaluating signal 1002. For example, near-by muscles when moved can generate EMGs that can be picked up from the leads.

In some examples, signal 1002 may contain features from which to make a determination that the patient moved at some time or times between times T0 and T3. For example, it may be known that stimulation-evoked signals do not cause the relatively large signal amplitude, or the large amplitude for that long of a period of time, as shown in signal 1002 between times T0 and T3. IMD 200A may then determine that signal 1002, at least between times T0 and T3, is low quality. Signal 1002 may contain stimulation-evoked signals, but they may be indistinguishable from signal 1002 between times T0 and T3 because they may be overwhelmed by signals received due to artifacts such as patient movement.

In some examples, IMD 200A may determine a plurality of qualities of signal 1002, e.g., at a plurality of times. In other words, IMD 200A may determine that the quality of a stimulation-evoked signal changes over time. By way of example, IMD 200A may determine that at least a portion of signal 1002 is of low quality because of an artifact for at least a portion of time between T0 and T4, e.g., due to patient movement at times T1 and T2. IMD 200A may determine a time at which the artifact ends, e.g., T4, and consequently a time at which the quality of signal 1002 may change, e.g., T4. IMD 200A may determine that the quality of signal 1002 is “medium” between times T4 and T3 because there may be uncertainty as to whether features of signal 1002 at or near the determined end time T4 of the artifact are due to signal responses or possible after-effects of the cause of artifact, e.g., patient movement in the current example. For example, IMD 200A may determine a signal feature based on a peak at time T5, but T5 may be close to time T4 and it may be an increased uncertainty as to whether the peak at time T5 is also at least partially an artifact, e.g., a residual response of any patient movement after-effects, or a stimulation-evoked response of a signal source to delivered electrical stimulation. IMD 200A may determine that uncertainty due to the cause of the artifact between times T1 and T4 may be sufficiently reduced and/or eliminated by time T3, and may determine that the quality of signal 1002 between times T3 and T7 is “good.” For example, IMD 200A may determine that signal features such as the peak at time T6 may be a reliable indication of a stimulation-evoked response of a signal source.

FIG. 11 is a plot 1100 of example accompanying signal 1102, in accordance with one or more techniques of this disclosure. In the example shown, signal 1102 is a pressure sensor signal amplitude that varies in time in proportion to bladder fullness. In some examples, signal 1102 may be captured by a pressure sensor within a urethral catheter during cystometry filling, and in some examples signal 1102 may be captured by a bladder pressure sensor over multiple fill/void cycles. In some examples, bladder fullness may be determine based on signal 1102 and/or a time between determined voiding events. For example, IMD 200A may receive signal 1102 from a pressure sensor 222 and determine that peaks 1104-1114 are voiding events (or are otherwise indicative of bladder activity), and that signal features 1116 includes artifacts. In some examples, IMD 200A may determine the quality of a stimulation-evoked signal (not shown) sensed contemporaneously with signal 1102 based on determined voiding events 1104-1114. IMD 200A may determine one or more qualities at one or more times of the contemporaneously sensed stimulation-evoked signal based on signal 1102, e.g., and determined voiding events 1104-1114.

FIG. 12 is a plot 1200 of example accompanying signal 1202, in accordance with one or more techniques of this disclosure. In the example shown, signal 1202 is a pressure sensor signal amplitude that varies in time in proportion to bladder fullness. In some examples, signal 1202 may be captured over multiple fill/void cycles. In some examples, bladder fullness may be determine based on signal 1202 and/or a time between determined voiding events. For example, IMD 200A may receive signal 1202 from a pressure sensor 222 and determine that peaks 1204-1216 are voiding events. In some examples, IMD 200A may determine the quality of a stimulation-evoked signal (not shown) sensed contemporaneously with signal 1202 based on determined voiding events 1204-1216. IMD 200A may determine one or more qualities at one or more times of the contemporaneously sensed stimulation-evoked signal based on signal 1202, e.g., and determined voiding events 1204-1216. In some examples, accompanying signal 1202 may be, or may be substantially similar to, an impedance sensor signal amplitude.

FIG. 13 is a plot 1300 of example accompanying signals 1320, 1330, and 1340, in accordance with one or more techniques of this disclosure. In the example shown, signals 1320-1340 are electro-ureterogram (EUG) sensor signal amplitudes that may vary in time in proportion to bladder fullness. In some examples, signals 1320-1340 may be captured over multiple fill/void cycles. In some examples, bladder fullness may be determine based on signals 1320-1340 and/or a time between determined voiding events. For example, IMD 200A may receive signals 1320-1340 from an EUG sensor 222 and determine that peaks 1302-1316 arise from peristalsis of the ureter events (e.g., filling of the bladder by squeezing boluses of urine from the kidney into the bladder). In some examples, IMD 200A may determine the quality of a stimulation-evoked signal (not shown) sensed contemporaneously with signal 1202 based on determined voiding events 1302-1316. IMD 200A may determine one or more qualities at one or more times of the contemporaneously sensed stimulation-evoked signal based on signals 1320-1340, e.g., and determined voiding events 1302-1316. In some examples, signals 1320 and 1330 may be from an electrode placed on an outer wall of the ureter and signal 1340 may be from a pressure sensor within the lumen of the ureter.

FIG. 14 is a plot 1400 of example accompanying signals 1402, 1404, in accordance with one or more techniques of this disclosure. In the example shown, signals 1402, 1404 are heart rate monitor sensor signal amplitudes that vary in time in proportion to the heart rate of the patient, e.g., from which a physiological state may be determined and an activity level and/or movement of the patient may be inferred. In the example shown, signal 1402 may correspond to a “fast” heart rate based on the frequency of pulses 1406, and signal 1404 may correspond to a “slow” heart rate based on the frequency of pulses 1408. In some examples, IMD 200A may determine the quality of a stimulation-evoked signal (not shown) sensed contemporaneously with signal 1402 or 1404, and may determine a quality of the contemporaneously sensed stimulation-evoked signal based on signals 1402 or 1404, e.g., and determined heart rates. For example, IMD 200A may determine the stimulation-evoked signal to be a “low” quality signal based on accompanying signal 1402, e.g., because IMD 200A may determine that the patient was either moving or the physiological state of an increased heart rate negatively affects the certainty of the stimulation-evoked signal and/or determination of its features. In another example, IMD 200A may determine the stimulation-evoked signal to be a “high” quality signal based on accompanying signal 1404, e.g., because IMD 200A may determine that the patient was either not moving or the physiological state of “resting” heart rate does not negatively affect the certainty of the stimulation-evoked signal and/or determination of its features.

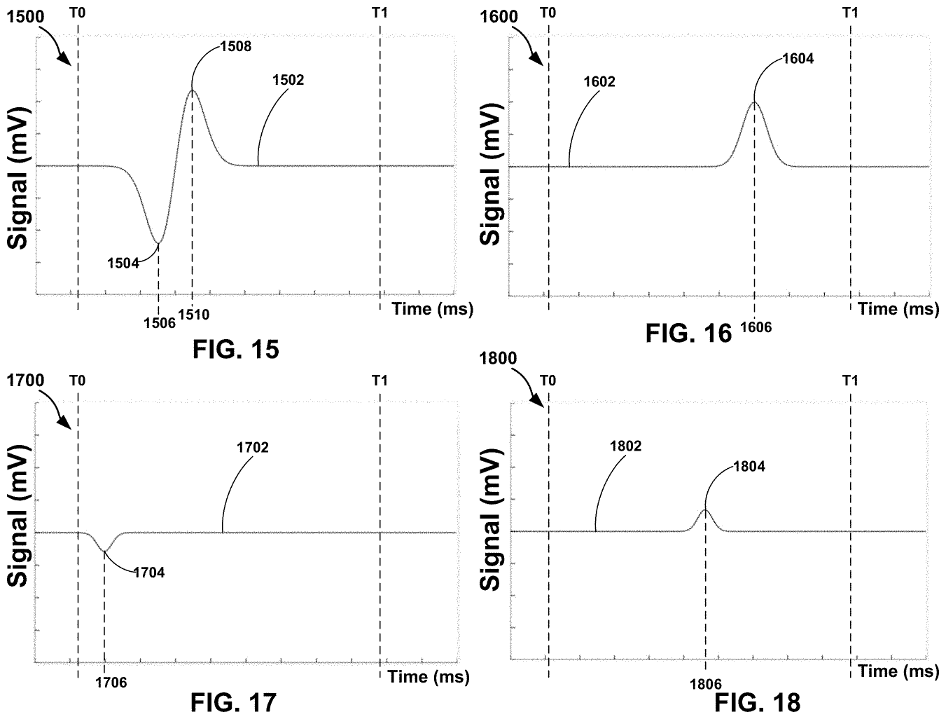

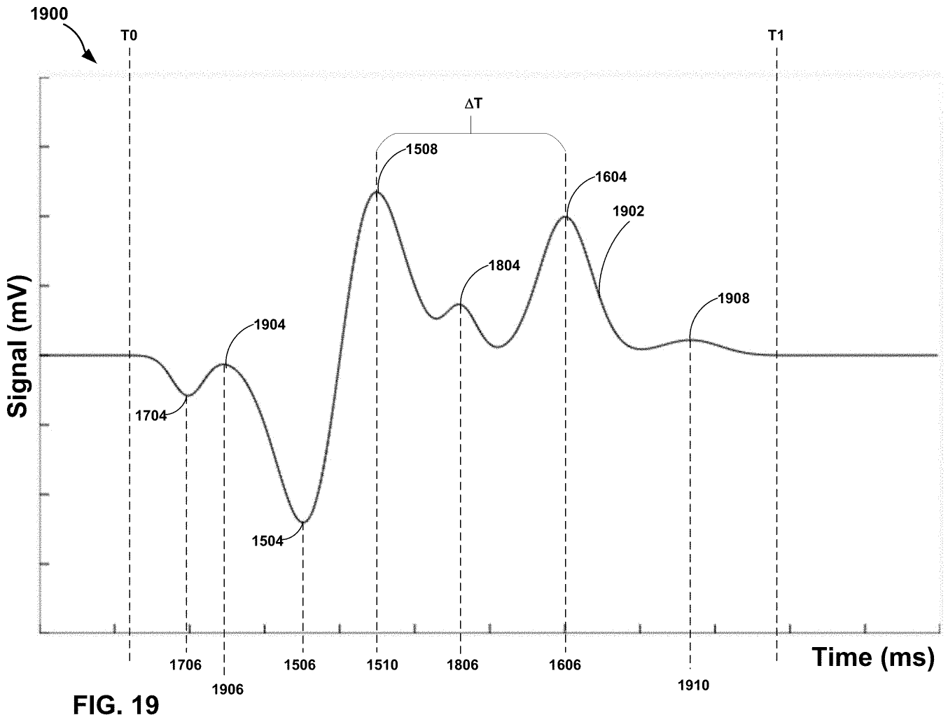

FIGS. 15-18 are plots of example stimulation-evoked signals and FIG. 19 is an example stimulation-evoked signal or composite stimulation-evoked signal, and are described together below. In the specific examples of FIGS. 15-19 below, each signal plotted represent a voltage amplitude of a circuit including an electrode 232 that varies in time in proportion to a time-varying electric field sensed by the electrode 232. The time-varying field in the examples shown is caused by one or more signal sources, e.g., nerve, muscle, or other tissue, of a patient in response to electrical stimulation. However, FIGS. 15-19 may generally represent one or more other quantities. In some examples, each signal plot may represent an amplitude as a function of time of a sensed quantity over time, the quantity varying in proportion to a physiological response of a signal source. In some examples, the quantity is an amplitude measured by a sensor. For example, the amplitude may be a voltage and/or current that varies in time according to an amplitude of an electric field and/or potential emitted and/or induced by a signal source. In some examples, the amplitude may be a displacement, a pressure, accelerometer data, a sound, e.g., such as an MMG signal. In some examples, composite stimulation-evoked signal 1902 described below may be a composite of sensed quantities from a plurality of sources sensed by a plurality of sensors, e.g., combined amplitudes as a function of time from two or more different sensors sensing two or more different quantities from one or more different signal sources that respond to the same electrical stimulation at or near the same time or within a period of time (e.g., a sensing “time window”). In some examples, two sensors may sense two different quantities from the same signal source, e.g., an EMG and an MMG of a muscle response. In other examples, composite stimulation-evoked signal 1902 may be a composite of a sensed quantity, e.g., an electric field and/or potential, from a plurality of signal sources sensed by the same sensor, e.g., an electrode 232 sensing a varying electric field that is a superposition of a plurality of electric fields caused by a plurality of signal sources responding to electrical stimulation within a sensing time window.

FIG. 15 is a plot 1500 of an example stimulation-evoked signal 1502, in accordance with one or more techniques of this disclosure. In the example shown, signal 1502 is a voltage amplitude that varies in time in proportion to a time-varying electric field sensed by an electrode 232, the time-varying electric field caused by a signal source in response to electrical stimulation. In the example shown, time T0 corresponds to a time at which electrical stimulation of a nerve or muscle ceases, e.g., is turned off, and time T1 corresponds to the ending time of the sensing time window, e.g., the sensing time window is the difference between T1 and T2. In some examples, signal 1502 may have a signal length in time that is equal to the time window, e.g., the physiological response of the signal source emits a detectable quantity (e.g., electric field) that lasts for the length of time the time window. In other examples, the signal length of signal 1502 may be less than the time window. Generally, the time window may be chosen based on signal length, e.g., time T0 may be chosen to be the time at which electrical stimulation ceases and time T1 may be chosen based on the time-length of the sensed signal, e.g., any of 1502, 1602, 1702, 1802, and/or 1902. In the examples of FIGS. 15-19, T1 chosen based on an exemplary time-length of signal 1902 and is shown on each of plots 1500-1900 for reference. In some examples, the length of stimulation-evoked signals 1502-1902 may be relatively long, e.g., 5 ms, 10 ms, 15 ms, 20 ms, 30 ms, or longer.

In the example shown, signal 1502 includes valley 1504 (which may be considered a “peak” with a negative amplitude and may be simply referred to as a “peak” herein) at time 1506 and peak 1508 at time 1510. In the example shown, signal 1502 may be a stimulation-evoked signal of a neural response of certain fibers of a nerve to electrical stimulation.

FIG. 16 is a plot 1600 of another example stimulation-evoked signal 1602, in accordance with one or more techniques of this disclosure. In the example shown, signal 1602 is a voltage amplitude that varies in time in proportion to a time-varying electric field sensed by an electrode 232, the time-varying electric field caused by a signal source in response to electrical stimulation. In the example shown, signal 1602 includes peak 1604 at time 1606. In the example shown, signal 1602 may be a stimulation-evoked signal of an EMG of a muscle in response to electrical stimulation.

FIG. 17 is a plot 1700 of another example stimulation-evoked signal 1702, in accordance with one or more techniques of this disclosure. In the example shown, signal 1702 is a voltage amplitude that varies in time in proportion to a time-varying electric field sensed by an electrode 232, the time-varying electric field caused by a signal source in response to electrical stimulation. In the example shown, signal 1702 includes valley 1704 at time 1706. In the example shown, signal 1702 may be a stimulation-evoked signal of a neural response of certain fibers of a nerve to electrical stimulation.

FIG. 18 is a plot 1800 of another example stimulation-evoked signal 1802, in accordance with one or more techniques of this disclosure. In the example shown, signal 1802 is a voltage amplitude that varies in time in proportion to a time-varying electric field sensed by an electrode 232, the time-varying electric field caused by a signal source in response to electrical stimulation. In the example shown, signal 1802 includes peak 1804 at time 1806. In the example shown, signal 1802 may be a stimulation-evoked signal of a neural response of one or more fibers of a nerve or an EMG of a muscle in response to electrical stimulation.

FIG. 19 is a plot of an example composite stimulation-evoked signal, in accordance with one or more techniques of this disclosure. In the example shown, signal 1902 is a voltage amplitude that varies in time in proportion to a time-varying electric field sensed by an electrode 232, the time-varying electric field caused by a plurality of signal sources in response to electrical stimulation. For example, signal 1902 may be a composite of signals 1502-1802. Although not shown, signal 1902 may include other peaks, features, artifacts, and/or noise. For example, electrode 232 may sense signal 1902 but not signals 1502-1802, which are illustrated for as individual components of composite signal 1902 for clarity.

In the example shown, composite stimulation-evoked signal 1902 includes peaks 1504, 1508, 1604, 1704, 1804, and 1904 and 1908 occurring at times 1506, 1510, 1606, 1706, 1806, and 1906 and 1910, respectively. In the example shown, peak 1904 may correspond to a combination of two or more stimulation-evoked signals from one or more signal sources. In other words, peak 1904 may not be a peak caused by a single signal, but rather is a result of the combination of signals 1502 and 1702. In some examples, a composite stimulation-evoked signal 1902 comprises two or more signals generated from one or more signal sources (e.g., in response to the electrical stimulation). Peak 1908 may be a stimulation-evoked signal of an EMG of a muscle in response to electrical stimulation, e.g., a second contraction of the same muscle of peak 1604 or a different muscle.

In some examples, IMD 200A may determine a plurality of features of signal 1902. For example, IMD 200A, external programmer 24, or another device such as a computing device, may receive signal 1902 and determine one or peaks 1504, 1508, 1604, 1704, 1804, 1904 and 1908, the corresponding times of the peaks, latency between one or more peaks such as ΔT between peak 1508 and 1604, the widths and areas of any of the above peaks, the frequency and/or spectral content of signal 1902, or any other signal feature, e.g., derivable via signal processing and/or digital signal processing.

In some examples, one or more determined feature may correspond to, and may be correlated with, the efficacy of stimulation therapy, e.g., a reduction in over-active bladder, bladder incontinence, bowel incontinence, fecal incontinence, or the like. For example, peak 1504 may relate to an electrical stimulation response of certain fibers of a nerve to electrical stimulation, peak 1604 may relate to an EMG of a muscle, and peak 1704 may relate to an electrical stimulation response of A fibers of a nerve which may relate to sensory and motor information. In some examples, improved and or optimal electrical stimulation therapy may be electrical stimulation that excites certain fibers of the nerves while reducing/minimizing excitation of certain other fibers, e.g., such that peak 508 is increased and peak 1704 is decreased. For example, a system may determine that leads 230 may be moved and/or stimulation parameters settings 242 may be adjusted to increase peak 1508 (e.g., increase excitation of certain fibers of a nerve) while also decreasing peak 1704 (e.g., reducing valley 1704 or making peak 1704 less negative, representing a decrease of the excitation of certain other fibers of a nerve).

As another example, improved and or optimal electrical stimulation therapy may be electrical stimulation that reduces/minimizes fiber excitation of some fibers while increasing excitation of other nerve fibers and muscle contraction, e.g., the EMG response of a muscle. For example, a system may determine that leads 230 may be moved and/or stimulation parameters settings 242 may be adjusted to increase peak 1704 (e.g., increase valley 1704 or make peak 1704 more negative, representing an increase of the excitation of certain fibers of a nerve) while increasing peak 1604 (e.g., increasing the response and corresponding EMG of a muscle) and decreasing peak 1508 (e.g., decreasing excitation of other fibers of a nerve).

The following numbered examples may illustrate one or more aspects of this disclosure:

Example 1: A method includes: delivering, via an electrical stimulation device, one or more electrical stimulation signals to a patient; sensing one or more stimulation-evoked signals that are evoked by stimulation of nerves or muscles of the patient due to the delivery of the one or more electrical stimulation signals; determining a quality of the one or more stimulation-evoked signals; and outputting, based on the quality of the one or more sensed stimulation-evoked signals being below a quality threshold, one or more instructions to improve the quality of one or more subsequent stimulation-evoked signals.

Example 2: The method of example 1, wherein delivering the one or more electrical stimulation signals to the patient for bladder or bowel control comprises delivering the one or more electrical stimulation signals to the patient for at least one of a pelvic health disorder, bladder dysfunction, retention, overactive bladder, urgency, urgency frequency, urinary incontinence, bladder incontinence, fecal incontinence, stress urinary incontinence, urinary retention, sexual dysfunction, obesity, gastroparesis, intractable constipation, pelvic pain, chronic pain, bladder pain syndrome, irritable bowel syndrome, inflammatory bowel disease, interstitial cystitis, neurogenic bowel, neurogenic bladder, neurological disorders, tremor, Parkinson's disease, epilepsy, multiple sclerosis, stroke, spinal cord injury, or neuropathy.

Example 3: The method of any one of examples 1-2, wherein the one or more electrical stimulation doses are delivered to at least one of a sacral nerve, a tibial nerve, a saphenous nerve, a pudendal nerve, or a sciatic nerve.

Example 4: The method of any one of examples 1-3, wherein the patient is in a non-clinic location.

Example 5: The method of any one of examples 1-4, where determining the quality of the one or more sensed stimulation-evoked signals comprises determining at least one of a signal-to-noise ratio (SNR), one or more signal artifacts, or determining that a patient is in a predetermined posture at the time the one or more electrical stimulation signals are delivered and/or at the time the one or more stimulation-evoked signals are sensed.

Example 6: The method of example 5, wherein one or more signal artifacts include at least one of a motion artifact, an electrical interference, a cardiac artifact, a bowel fullness, a bladder fullness, a spurious EMG, a standard deviation less than a threshold standard deviation, a variance less than a threshold variance, or a SNR greater than a threshold SNR.

Example 7: The method of any one of examples 5-6, wherein the predetermined posture comprises at least one of a patient movement or a patient position, the patient position comprising one of sitting down, standing, or lying down.

Example 8: The method of any one of examples 1-7, wherein the one or more stimulation-evoked signals comprises at least one of an electromyographic (EMG) signal an evoked compound action potential (ECAP), a neural network excitability, or a muscular network excitability.

Example 9: The method of example 8, wherein the predetermined posture is a first predetermined posture, wherein the one or more instructions comprises at least one of directing the patient to be in a second predetermined posture different from the first predetermined posture during subsequent delivery of electrical stimulation, directing at least one of the patient or a clinician to change an electrical stimulation parameter, directing the electrical stimulation device to change an electrical stimulation parameter, or waiting for a predetermined amount of time before subsequent delivery of electrical stimulation.

Example 10: The method of any one of examples 1-9, further includes determining, based on the quality of the one or more sensed stimulation-evoked signals, an efficacy of a stimulation therapy program; and outputting the determined efficacy.

Example 11: The method of any of examples 1 through 10, wherein the one or more stimulation-evoked signals comprise a composite stimulation-evoked signal comprising two or more stimulation-evoked signals generated by one or more signal sources in response to the one or more electrical stimulation signals.

Example 12: A system includes: at least one electrode configured to deliver the electrical stimulation to a patient; and a device including processing circuitry configured to: deliver one or more electrical stimulation signals to a patient; sense one or more stimulation-evoked signals that are evoked by stimulation of nerves or muscles of the patient due to the delivery of the one or more electrical stimulation signals; determine a quality of the one or more stimulation-evoked signals; and output, based on the quality of the one or more sensed stimulation-evoked signals being below a quality threshold, one or more instructions to improve the quality of one or more subsequent stimulation-evoked signals.

Example 13: The system of example 12, wherein delivering the one or more electrical stimulation signals to the patient comprises delivering the one or more electrical stimulation signals to the patient for at least one of a pelvic health disorder, bladder dysfunction, retention, overactive bladder, urgency, urgency frequency, urinary incontinence, bladder incontinence, fecal incontinence, stress urinary incontinence, urinary retention, sexual dysfunction, obesity, gastroparesis, intractable constipation, pelvic pain, chronic pain, bladder pain syndrome, irritable bowel syndrome, inflammatory bowel disease, interstitial cystitis, neurogenic bowel, neurogenic bladder, neurological disorders, tremor, Parkinson's disease, epilepsy, multiple sclerosis, stroke, spinal cord injury, or neuropathy.

Example 14: The system of any one of examples 12-13, wherein the one or more electrical stimulation signals are delivered to at least one of a sacral nerve, a tibial nerve, a saphenous nerve, a pudendal nerve, or a sciatic nerve.

Example 15: The system of any one of examples 12-14, wherein the system is configured to be used with a patient in a non-clinic location.

Example 16: The system of any one of examples 12-15, where determining the quality of the one or more sensed stimulation-evoked signals comprises determining at least one of a signal-to-noise ratio (SNR), one or more signal artifacts, or determining that a patient is in a predetermined posture at the time the one or more electrical stimulation signals are delivered and/or at the time the one or more stimulation-evoked signals are sensed.

Example 17: The system of example 16, wherein one or more signal artifacts include at least one of a motion artifact, an electrical interference, a cardiac artifact, a bowel fullness, a bladder fullness, a spurious EMG, a standard deviation less than a threshold standard deviation, a variance less than a threshold variance, or a SNR greater than a threshold SNR.

Example 18: The system of any one of examples 16-17, wherein the predetermined posture comprises at least one of a patient immobility or a patient position, the patient position comprising one of sitting down or lying down.

Example 19: The system of any one of examples 12-18, wherein the one or more stimulation-evoked signals comprises at least one of an electromyographic (EMG) signal, an evoked compound action potential (ECAP), a neural network excitability, or a muscular network excitability.

Example 20: The system of example 19, wherein the one or more instructions comprises at least one of directing the patient to be in the predetermined posture during delivery of electrical stimulation, directing at least one of the patient or a clinician to change an electrical stimulation parameter, directing an electrical stimulation device configured to control the at least one electrode to change an electrical stimulation parameter, or waiting for a predetermined amount of time before subsequent delivery of electrical stimulation.

Example 21: A computer readable medium including instructions that when executed cause one or more processors to: cause an electrical stimulation device to: deliver one or more electrical stimulation doses to a patient; receive one or more sensed stimulation-evoked signals that are evoked by stimulation of nerves or muscles of the patient due to the delivery of the one or more electrical stimulation signals; determine a quality of the one or more sensed stimulation-evoked signals; and output, based on the quality of the one or more sensed stimulation-evoked signals being below a quality threshold, one or more instructions to improve the quality of one or more subsequent stimulation-evoked signals.

The techniques described in this disclosure may be implemented, at least in part, in hardware, software, firmware or any combination thereof. For example, various aspects of the described techniques may be implemented within processing circuitry, which may include one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also form one or more processors or processing circuitry configured to perform one or more of the techniques of this disclosure.

Such hardware, software, and firmware may be implemented, and various operation may be performed within same device, within separate devices, and/or on a coordinated basis within, among or across several devices, to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components. Processing circuitry described in this disclosure, including a processor or multiple processors, may be implemented, in various examples, as fixed-function circuits, programmable circuits, or a combination thereof. Fixed-function circuits refer to circuits that provide particular functionality with preset operations. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive stimulation parameters or output stimulation parameters), but the types of operations that the fixed-function circuits perform are generally immutable. In some examples, one or more of the units may be distinct circuit blocks (fixed-function or programmable), and in some examples, one or more of the units may be integrated circuits.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

What is claimed is:

1. A method comprising:
delivering, via an electrical stimulation device, one or more electrical stimulation signals to a patient;
sensing one or more stimulation-evoked signals that are evoked by stimulation of nerves or muscles of the patient due to the delivery of the one or more electrical stimulation signals;
determining a quality of the one or more stimulation-evoked signals; and
outputting, based on the quality of the one or more sensed stimulation-evoked signals being below a quality threshold, one or more instructions to improve the quality of one or more subsequent stimulation-evoked signals.

2. The method of claim 1, wherein delivering the one or more electrical stimulation signals to the patient comprises delivering the one or more electrical stimulation signals to the patient for at least one of a pelvic health disorder, bladder dysfunction, retention, overactive bladder, urgency, urgency frequency, urinary incontinence, bladder incontinence, fecal incontinence, stress urinary incontinence, urinary retention, sexual dysfunction, obesity, gastroparesis, intractable constipation, pelvic pain, chronic pain, bladder pain syndrome, irritable bowel syndrome, inflammatory bowel disease, interstitial cystitis, neurogenic bowel, neurogenic bladder, neurological disorders, tremor, Parkinson's disease, epilepsy, multiple sclerosis, stroke, spinal cord injury, or neuropathy.

3. The method of claim 1, wherein the one or more electrical stimulation doses are delivered to at least one of a sacral nerve, a tibial nerve, a saphenous nerve, a pudendal nerve, or a sciatic nerve.

4. The method of claim 1, wherein the patient is in a non-clinic location.

5. The method of claim 1, where determining the quality of the one or more sensed stimulation-evoked signals comprises determining at least one of a signal-to-noise ratio (SNR), one or more signal artifacts, or determining that a patient is in a predetermined posture at the time the one or more electrical stimulation signals are delivered and/or at the time the one or more stimulation-evoked signals are sensed.

6. The method of claim 5, wherein one or more signal artifacts include at least one of a motion artifact, an electrical interference, a cardiac artifact, a bowel fullness, a bladder fullness, a spurious EMG, a standard deviation less than a threshold standard deviation, a variance less than a threshold variance, or a SNR greater than a threshold SNR.

7. The method of claim 5, wherein the predetermined posture comprises at least one of a patient movement or a patient position, the patient position comprising one of sitting down, standing, or lying down.

8. The method of claim 1, wherein the one or more stimulation-evoked signals comprises at least one of an electromyographic (EMG) signal, an evoked compound action potential (ECAP), a neural network excitability, or a muscular network excitability.

9. The method of claim 8, wherein the predetermined posture is a first predetermined posture, wherein the one or more instructions comprises at least one of directing the patient to be in a second predetermined posture different from the first predetermined posture during subsequent delivery of electrical stimulation, directing at least one of the patient or a clinician to change an electrical stimulation parameter, directing the electrical stimulation device to change an electrical stimulation parameter, or waiting for a predetermined amount of time before subsequent delivery of electrical stimulation.

10. The method of claim 1, further comprising:
determining, based on the quality of the one or more sensed stimulation-evoked signals, an efficacy of a stimulation therapy program; and
outputting the determined efficacy.

11. The method of claim 1, wherein the one or more stimulation-evoked signals comprise a composite stimulation-evoked signal comprising two or more stimulation-evoked signals generated by one or more signal sources in response to the one or more electrical stimulation signals.

12. A system comprising:
at least one electrode configured to deliver the electrical stimulation to a patient; and
a device comprising processing circuitry configured to:
deliver one or more electrical stimulation signals to a patient;
sense one or more stimulation-evoked signals that are evoked by stimulation of nerves or muscles of the patient due to the delivery of the one or more electrical stimulation signals;
determine a quality of the one or more stimulation-evoked signals; and
output, based on the quality of the one or more sensed stimulation-evoked signals being below a quality threshold, one or more instructions to improve the quality of one or more subsequent stimulation-evoked signals.

13. The system of claim 12, wherein delivering the one or more electrical stimulation signals to the patient comprises delivering the one or more electrical stimulation signals to the patient for at least one of a pelvic health disorder, bladder dysfunction, retention, overactive bladder, urgency, urgency frequency, urinary incontinence, bladder incontinence, fecal incontinence, stress urinary incontinence, urinary retention, sexual dysfunction, obesity, gastroparesis, intractable constipation, pelvic pain, chronic pain, bladder pain syndrome, irritable bowel syndrome, inflammatory bowel disease, interstitial cystitis, neurogenic bowel, neurogenic bladder, neurological disorders, tremor, Parkinson's disease, epilepsy, multiple sclerosis, stroke, spinal cord injury, or neuropathy.

14. The system of claim 12, wherein the one or more electrical stimulation signals are delivered to at least one of a sacral nerve, a tibial nerve, a saphenous nerve, a pudendal nerve, or a sciatic nerve.

15. The system of claim 12, wherein the system is configured to be used with a patient in a non-clinic location.

16. The system of claim 12, where determining the quality of the one or more sensed stimulation-evoked signals comprises determining at least one of a signal-to-noise ratio (SNR), one or more signal artifacts, or determining that a patient is in a predetermined posture at the time the one or more electrical stimulation signals are delivered and/or at the time the one or more stimulation-evoked signals are sensed.

17. The system of claim 16, wherein one or more signal artifacts include at least one of a motion artifact, an electrical interference, a cardiac artifact, a bowel fullness, a bladder fullness, a spurious EMG, a standard deviation less than a threshold standard deviation, a variance less than a threshold variance, or a signal-to-noise ratio (SNR) greater than a threshold SNR.

18. The system of claim 16, wherein the predetermined posture comprises at least one of a patient immobility or a patient position, the patient position comprising one of sitting down or lying down.

19. The system of claim 12, wherein the one or more stimulation-evoked signals comprises at least one of an electromyographic (EMG) signal, an evoked compound action potential (ECAP), a neural network excitability, or a muscular network excitability.

20. The system of claim 19, wherein the one or more instructions comprises at least one of directing the patient to be in the predetermined posture during delivery of electrical stimulation, directing at least one of the patient or a clinician to change an electrical stimulation parameter, directing an electrical stimulation device configured to control the at least one electrode to change an electrical stimulation parameter, or waiting for a predetermined amount of time before subsequent delivery of electrical stimulation.

21. A computer readable medium comprising instructions that when executed cause one or more processors to:
cause an electrical stimulation device to deliver one or more electrical stimulation doses to a patient;
receive one or more sensed stimulation-evoked signals that are evoked by stimulation of nerves or muscles of the patient due to the delivery of the one or more electrical stimulation signals;
determine a quality of the one or more sensed stimulation-evoked signals; and
output, based on the quality of the one or more sensed stimulation-evoked signals being below a quality threshold, one or more instructions to improve the quality of one or more subsequent stimulation-evoked signals.
